(19) United States
(12) Patent Application Publication
(10) Pub. No.: US 2025/0134964 A1
(43) Pub. Date: May 1, 2025

(54) METHOD FOR INDUCING WEIGHT LOSS IN A TYPE 2 DIABETES MELLITUS PATIENT

(71) Applicant: SANOFI-AVENTIS DEUTSCHLAND GMBH, Frankfurt am Main (DE)

(72) Inventors: Elisabeth NIEMOELLER, Frankfurt am Main (DE); Louise SILVESTRE, Paris (FR); Gabor BOKA, Paris (FR); Patrick MIOSSEC, Paris (FR)

(21) Appl. No.: 18/987,056

(22) Filed: Dec. 19, 2024

Related U.S. Application Data
(63) Continuation of application No. 17/695,343, filed on Mar. 15, 2022.
Application No. 17/695,343 is a continuation of application No. 17/496,894, filed on Oct. 8, 2021, now abandoned.
Application No. 17/496,894 is a continuation of application No. 16/727,848, filed on Dec. 26, 2019, now abandoned.
Application No. 16/727,848 is a continuation of application No. 15/960,488, filed on Apr. 23, 2018, now abandoned.
Application No. 15/960,488 is a continuation of application No. 15/730,033, filed on Oct. 11, 2017, now abandoned.
Application No. 15/730,033 is a division of application No. 13/467,707, filed on May 9, 2012, now Pat. No. 9,821,032.

(30) Foreign Application Priority Data
May 13, 2011 (EP) 11166111.2

(51) Int. Cl. A61K 38/26; A61K 31/155; A61K 38/28

(52) U.S. Cl. CPC A61K 38/26; A61K 31/155; A61K 38/28; A61K 2300/00

(57) ABSTRACT

The present invention refers to a pharmaceutical combination for use in glycemic control in diabetes type 2 patients.

Subject of the present invention is a pharmaceutical combination for use in glycemic controlin diabetes type 2 patients, said combination-comprising (a) desPro³⁶Exendin-4(1-39)-Lys₆-NH₂ (AVE0010, lixisenatide) or/and a pharmaceutically acceptable salt thereof, (b) a basal insulin or/and a pharmaceutically acceptable salt thereof, and (c) optionally metformin or/and a pharmaceutically acceptable salt thereof. A further subject of the present invention is a pharmaceutical combination for use in the prevention of hypoglycemia, said combination comprising (a) desPro³⁶Exendin-4(1-39)-Lys6-NH2 or/and a pharmaceutically acceptable salt thereof, (b) a basal insulin or/and a pharmaceutically acceptable salt thereof, and (c) optionally metformin or/and a pharmaceutically acceptable salt thereof. Another subject of the present invention is a pharmaceutical combination for use in inducing weight loss in diabetes type 2 patients or/and for preventing weight gain in diabetes type 2 patients, said combination comprising (a) desPro³⁶Exendin-4(1-39)-Lys6-NH2 or/and a pharmaceutically acceptable salt thereof, (b) a basal insulin or/and a pharmaceutically acceptable salt thereof, and (c) optionally metformin or/and a pharmaceutically acceptable salt thereof.

In a healthy person the release of insulin by the pancreas is strictly coupled to the concentration of blood glucose. An increased level of blood glucose, as appears after meals, is rapidly counterbalanced by a respective increase in insulin secretion. In fasting condition the plasma insulin level drops to a basal value which is sufficient to ensure the continuous supply of glucose to insulin-sensitive organs and tissues and to keep the hepatic glucose production at a low level at night.

In contrast to diabetes type 1, there is not a lack of insulin in diabetes type 2 but in many cases, particularly in progressive cases, the treatment with insulin is regarded as the most suitable therapy, if required in combination with orally administered anti-diabetic drugs.

An increased glucose level in the blood over several years without initial symptoms represents a significant health risk. It could clearly be shown by the large-scale DCCT study in the USA (The Diabetes Control and Complications Trial Research Group (1993) N. Engl. J. Med. 329, 977-986) that chronically increased levels of blood glucose are a main reason for the-development of diabetes complications. Examples for diabetes complications are micro and macrovascular damages that possibly manifest themselves in retinopathies, nephropathies or neuropathies and lead to blindness, renal. failure and the loss of extremities and are accompanied by an increased risk of cardiovascular diseases. It can thus be concluded that an improved. therapy of diabetes primarily has to aim keeping blood glucose in the physiological range as closely as possible.

A particular risk exists for overweight patients suffering from diabetes type 2, e.g. patients with a body mass index (BMI) >30. In these patients the risks of diabetes overlap with the risks of overweight, leading e.g. to an increase of cardiovascular diseases compared to diabetes type 2 patients being of a normal weight. Thus, it is particularly necessary to treat diabetes in these patients while reducing the overweight.

Metformin is a biguanide hypoglycemic agent used in the treatment of non-insulin-dependent diabetes mellitus (diabetes mellitus type 2) not responding to dietary modification. Metformin improves glycemic control by improving insulin sensitivity and decreasing intestinal absorption of glucose. Metformin is usually administered orally. However, control diabetes mellitus type 2 in obese patients by metformin may be insufficient. Thus, in these patients, additional measures for controlling diabetes mellitus type 2 may be required.

Insulin is a polypeptide having 51 amino acid residues. Insulin consists of the A chain having 21 amino acid residues, and the B chain having 30 amino acid residues. The chains are coupled by 2 disulfide bridges. Insulin formulations have been used for a long time for therapy of diabetes mellitus type 1 and 2. Recently, insulin derivatives and insulin analogues have been used.

The compound desPro³⁶Exendin-4(1-39)-Lys₆-NH2 (AVE0010, lixisenatide) is a derivative of Exendin-4. AVE0010 is disclosed as SEQ ID NO:93 in WO 01/04156:

SEQ ID NO: 1: AVE0010 (44 AS)
H-G-E-G-T-F-T-S-D-L-S-K-Q-M-E-E-E-A-V-R-L-F-I-E-W-
L-K-N-G-G-P-S-S-G-A-P-.P-S-K-K-K-K-K-K-NH₂
SEQ ID NO: 2: Exendin-4 (39 AS)
H-G-E-G-T-F-T-S-D-L-S-K-Q-M-E-E-E-A-V-R-L-F-I-E-W-
L-K-N-G-G-P-S-S-G-A-P-P-P-S-NH₂

Exendins are a group of peptides which can lower blood glucose concentration. The Exendin analogue AVE0010 is characterised by C-terminal truncation of the native Exendin-4 sequence. AVE0010 comprises six C-terminal lysine residues not present in Exendin-4.

In the context of the present invention, AVE0010 includes pharmaceutically acceptable salts thereof. The person skilled in the art knows pharmaceutically acceptable salts of AVE0010. A preferred pharmaceutically acceptable salt of AVE0010 employed in the present invention is acetate.

In the Example of the present invention, it has been demonstrated that AVE0010 (Lixisenatide) in an add-on therapy to basal insulin and optionally metformin significantly improved glycemic control and decreased weight:
HbA1c was significantly decreased.
postprandial plasma glucose concentration was significantly improved with lixisenatide.
Significant weight loss was induced.
A significant decrease in daily basal insulin dose could be observed.

No significant increase in the incidence of hypoglycemia was. observed.

A first aspect of the present invention is a pharmaceutical combination for use in glycemic control in diabetes type 2 patients, said combination comprising
(a) desPro³⁶Exendin-4(1-39)-Lys₆-NH₂ or/and a pharmaceutically acceptable salt thereof,
(b) a basal insulin or/and a pharmaceutically acceptable salt thereof, and
(c) optionally metformin or/and a pharmaceutically acceptable salt thereof.

As demonstrated by the Example disclosed herein, the combination as described herein can be used for improving glycemic control. In the present invention, “improvement of glycemic control” or “glycemic control” in particular refers to improvement of postprandial plasma glucose concentration, improvement of fasting plasma glucose concentration, or/and improvement of the HbA_{1c} value.

Metformin is the international nonproprietary name of 1,1-dimethylbiguanide (CAS Number 657-24-9). In the present invention, the term “metformin” includes any pharmaceutically acceptable salt thereof.

In the present invention, metformin may be administered orally. The skilled person knows formulations of metformin suitable for treatment of diabetes type 2 by oral administration. Metformin may be administered to a subject in need thereof, in an amount sufficient to induce a therapeutic effect. Metformin may be administered in a dose of at least 1.0 g/day or at least 1.5 g/day. For oral administration, metformin may be formulated in a solid dosage form, such as a tablet or pill. Metformin may be formulated with suitable pharmaceutically acceptable carriers, adjuvants, or/and auxiliary substances.

In the present invention, desPro³⁶Exendin-4(1-39)-Lys₆-NH₂ or/and a pharmaceutically acceptable salt may be administered in an add-on therapy to administration of a basal insulin and optionally metformin.

In the present invention, the terms “add-on”, “add-on treatment” and “add-on therapy” relate to treatment of diabetes mellitus type 2 with metformin, AVE0010 and the basal insulin. Metformin, AVE0010 and the basal insulin may be administered within a time interval of 24 h. Metformin, AVE0010 and the basal insulin each may be administered in a once-a-day-dosage. Metformin, AVE0010 and basal insulin may be administered by different administration routes. Metformin may be administered orally, and AVE0010 and the basal insulin may be administered parenterally.

In the present invention, the terms “add-on”, “add-on treatment” and “add-on therapy” also relate to treatment of diabetes mellitus type 2 with AVE0010 and the basal insulin. AVE0010 and the basal insulin may be administered within a time interval of 24 hAVE0010 and the basal insulin each may be administered in a once-a-day-dosage. AVE0010 and basal insulin may be administered parenterally.

In the present invention, “basal insulin” includes suitable pharmaceutically acceptable salts thereof. In the present invention, any basal insulin can be used. In particular, the basal insulin can be selected from insulin Glargine, Detemir, NPH, Lente, Ultralente, Novolin®, Humalog® and mixtures thereof. The mixture may comprise two different basal insulins. For example, a mixture comprising Detemir and Glargine, or a mixture comprising NPH and Novolin®, may be employed. Preferably, the basal insulin is insulin Glargine (Lantus®), or a mixture comprising insulin Glargine.

Insulin glargine (Lantus) is Gly(A21)-Arg(B31)-Arg(B32)-human insulin. In the present invention, insulin Glargine includes pharmaceutically acceptable salts thereof.

The basal insulin or/and a pharmaceutically acceptable salt thereof may be administered parenterally, e.g. by injection (such as by intramuscular or by subcutaneous injection). Suitable injection devices, for instance the so-called “pens” comprising a cartridge comprising the active ingredient, and an injection needle, are known. The basal insulin or/and a pharmaceutically acceptable salt thereof may be administered in a suitable amount, for instance in an amount in the range of 15 to 80 U per dose.

In the present invention, the basal insulin or/and a pharmaceutically acceptable salt thereof may be administered in a daily dose in the range of 15 to 80 U. Insulin glargine or/and a pharmaceutically acceptable salt thereof may be administered once daily, for example by one injection per day.

The skilled person knows formulations of basal insulin, including suitable pharmaceutically acceptable carriers; adjuvants or/and auxiliary substances.

In the present invention, the basal insulin or/and a pharmaceutically acceptable salt thereof may be provided in a liquid composition The skilled person knows liquid compositions of basal insulins suitable for parenteral administration.

In the present invention, the basal insulin or/and the pharmaceutically acceptable salt thereof may be administered to a subject in need thereof, in an amount sufficient to induce a therapeutic effect.

The subject to be treated by the medicament of the present invention suffering from diabetes type 2 may be a subject suffering from diabetes type 2, wherein diabetes type 2 is not adequately controlled by treatment with the basal insulin and optionally metformin alone, for instance with a dose of 15 to 80 U/day insulin for 3 months and optionally with a dose of at least 1.0 g/day metformin or at least 1.5 g/day metformin for 3 months. In the present invention, a subject the diabetes type 2 of which is not adequately controlled may have a HbA_{1c} value in the range of 7% to 10%.

The subject to be treated by the medicament of the present invention suffering from diabetes type 2 may be an obese subject In the present invention, an obese subject may have a body mass index of at least 30 kg/m².

The subject to be treated by the medicament of the present invention suffering from diabetes type 2 may have a normal body weight. In the present invention, a subject having normal body weight may have a body mass index in the range of 17 kg/m² to 25 kg/m², or 17 kg/m² to <30 kg/m²

The subject to be treated by the medicament of the present invention may be an adult subject. The subject may have an age of at least 18 years of may have an age in the range of 18 to 80 years, of 18 to 50 years, or 40 to 80 years, or 50 to 60 years. The subject may be younger than 50 years.

The subject to be treated by the medicament of the present invention may suffer from diabetes mellitus type 2 for at least 1 year or at least 2 years. In particular, in the subject to be treated, diabetes mellitus type 2 has been diagnosed at least 1 year or at least 2 years before onset of therapy by the. medicament of the present invention.

The subject to be treated may have a HbA_{1c} value of at least about 8% or at least about 7.5%. The subject may also have a HbA_{1c} value of about to about 10%. The example of the present invention demonstrates that treatment by AVE0010 results in a reduction of the HbA1c value in diabetes type 2 patients.

In yet another aspect of the present invention, the combination as described herein can be used for improving the HbA1c value in a patient suffering from diabetes type 2. Improving the HbA1c value means that the HbA_{1c} value is reduced below 6.5% or 7%, for example after treatment for at least one month, at least two months, or at least three months.

In yet another aspect of the present invention, the combination as described herein can be used for improving glucose tolerance in a patient suffering from diabetes type 2. Improving glucose tolerance means that the postprandial plasma glucose concentration is reduced by the active agent of the present invention. Reduction means in particular that the plasma glucose concentration reaches normoglycemic values or at least approaches these values.

In the present invention, normoglycemic value-s are blood glucose concentrations of in particular 60-140 mg/dl (corresponding to 3,3 bis 7,8 mM/L). This range refers in particular to blood glucose concentrations under fasting conditions and postprandial conditions.

The subject to be treated may have a 2 hours postprandial plasma glucose concentration of at least 10 mmol/L, at least 12 mmol/L, or at least 14 mmol/L. These plasma glucose concentrations exceed normoglycemic concentrations.

The subject to be treated may have a glucose excursion of at least 2 mmol/L, at least 3 mmol/L, at least 4 mmol/L or at least 5 mmol/L. In the present invention, the glucose excursion is in particular the difference of the 2 hours postprandial plasma glucose concentration and the plasma glucose concentration 30 minutes prior to a meal test

“Postprandial” is a term that is well known to a person skilled in the art of diabetology. The term “postprandial” describes in particular the phase after a meal or/and exposure to glucose under experimental conditions. In a healthy person this phase is characterised by an increase and subsequent decrease in blood glucose concentration. The term “postprandial” or “postprandial phase” typically ends up to 2 h after a meal or/and exposure to glucose.

The subject to be treated as disclosed herein may have a fasting plasma glucose concentration of at least 8 mmol/L, at least 8,5 mmol/L or at least 9 mmol/L. These plasma glucose concentrations exceed normoglycemic concentrations.

In another aspect of the present invention, the combination as described herein can be used for improving (i.e. reducing) fasting plasma glucose in a patient suffering from diabetes type 2. Reduction means in particular that the plasma glucose concentration reaches normoglycemic values or at least approaches these values.

A further aspect of the present invention is a method for improving glycemic control in diabetes type 2 patients, said method comprising administering desPro³⁶Exendin-4 (1-39)-Lys₆-NH₂ or/and a pharmaceutically acceptable salt thereof, in combination with metformin to a subject in need thereof. In particular, the combination as described herein may be administered. In the method of the present invention, the subject may be the subject defined herein.

The combination of the present invention can be used in the treatment of one or more of the medical indications described herein, for example in treatment of diabetes type 2 patients, or for conditions associated with diabetes type 2, such as improvement of glycemic control, reduction of the fasting plasma glucose concentration, for the improvement of glucose excursion, reduction of the postprandial plasma glucose concentration, improvement of glucose tolerance, improving the HbA_{1c} value, for the prevention of hypoglycaemia, for weight loss or/and prevention of weight gain.

In the present invention, desPro³⁶Exendin-4(1-39)-Lys₆-NH₂ or/and the pharmaceutically acceptable salt thereof may be administered to a subject in need thereof, in an amount sufficient to induce a therapeutic effect.

In the present invention, desPro³⁶Exendin-4(1-39)-Lys₆-NH₂ or/and the pharmaceutically acceptable salt thereof may be formulated with suitable pharmaceutically acceptable carriers, adjuvants, or/and auxiliary substances.

The compound desPro³⁶Exendin-4(1-39)-Lys₆-NH₂ or/and a pharmaceutically acceptable salt thereof may be administered parenterally, e.g. by injection (such as by intramuscular or by subcutaneous injection). Suitable injection devices, for instance the so-called “pens” comprising a cartridge comprising the active ingredient, and an injection needle, are known. The compound desPro³⁶Exendin-4(1-39)-Lys₆-NH₂ or/and a pharmaceutically acceptable salt thereof may be administered in a suitable amount, for instance in an amount in the range of 10 to 15 μg per dose or 15 to 20 μg per dose.

In the present invention, desPro³⁶Exendin-4(1-39)-Lys₆-NH₂ or/and a pharmaceutically acceptable salt thereof may be administered in a daily dose in the range of 10 to 20 μg, in the range of 10 to 15 μg, or in the range of 15 to 20 μg. DesPro³⁶Exendin-4(1-39)-Lys₆-NH₂ or/and a pharmaceutically acceptable salt thereof may be administered by one injection per day.

In the present invention, desPro³⁶Exendin-4(1-39)-Lys₆-NH₂ or/and a pharmaceutically acceptable salt thereof may be provided in a liquid composition. The skilled person knows liquid compositions of AVE0010 suitable for parenteral administration. A liquid composition of the present invention may have an acidic or a physiologic pH. An acidic pH preferably is in the range of pH 1-6.8, pH 3.5-6.8, or pH 3.5-5. A physiologic pH preferably is in the range of pH 2.5-8.5, pH 4.0-8.5, or pH 6.0-8.5. The pH may be adjusted by a pharmaceutically acceptable diluted acid (typically HCl) or pharmaceutically acceptable diluted base (typically NaOH).

The liquid composition comprising desPro³⁶Exendin-4(1-39)-Lys₆-NH₂ or/and a pharmaceutically acceptable salt thereof may comprise a suitable preservative. A suitable preservative may be selected from phenol, m-cresol, benzyl alcohol and p-hydroxyberizoic acid ester. A preferred preservative is m-cresol.

The liquid composition comprising desPro³⁶Exendin-4(1-39)-Lys₆-NH₂ or/and a pharmaceutically acceptable salt thereof may comprise a tonicity agent. A suitable tonicity agent may be selected from glycerol, lactose, sorbitol, mannitol, glucose, NaCl, calcium or magnesium containing compounds such as CaCl₂. The concentration of glycerol, lactose, sorbitol, mannitol and glucose may be in the range of 100-250 mM. The concentration of NaCl may be up to 150 mM. A preferred tonicity agent is glycerol.

The liquid composition comprising desPro³⁶Exendin-4(1-39)-Lys₆-NH₂ or/and a pharmaceutically acceptable salt thereof may comprise methionine from 0.5 μg/mL to 20 μg/mL, preferably from 1 μg/ml to 5 μg/ml. Preferably, the liquid composition comprises L-methionine.

Another aspect of the present invention is a pharmaceutical combination for use in inducing weight loss in diabetes type 2 patients or/and for preventing weight gain in diabetes type 2 patients, said combination comprising
(a) desPro³⁶ Exendin-4(1-39)-Lys₆-NH₂ or/and a pharmaceutically acceptable salt thereof,
(b) a basal insulin or/and a pharmaceutically acceptable salt thereof, and
(c) optionally metformin or/and a pharmaceutically acceptable salt thereof.

A further aspect of the present invention is a method for inducing weight loss in diabetes type 2 patients or/and for preventing weight gain in diabetes type 2 patients, said method comprising administering desPro³⁶Exendin-4(1-39)-Lys₆-NH₂ or/and a pharmaceutically acceptable salt thereof, in combination with metformin to a subject in need thereof. In particular, the combination as described herein may be administered. In the method of the present invention, the subject may be the subject defined herein.

Yet another aspect of the present invention is a pharmaceutical combination for use in the prevention of hypoglycaemia in diabetes mellitus type 2 patients, said combination comprising
(a) desPro³⁶Exe din-4(1-39)-Lys₆-NH₂ or/and a pharmaceutically acceptable salt thereof,
(b) a basal insulin or/and a pharmaceutically acceptable salt thereof, and
(c) optionally metformin or/and a pharmaceutically acceptable salt thereof.

In particular, the pharmaceutical combination is used for the prevention of symptomatic hypoglycaemia or/and severe symptomatic hypoglycaemia in a diabetes mellitus type 2 patient

In the present invention, hypoglycaemia is a condition wherein a diabetes mellitus type 2 patient experiences a plasma glucose concentration of below 60 mg/dl (or below 3.3 mmol/L), below 50 mg/dl, below 40 mg/dl, or below 36 mg/dL.

By the method of the present invention, hypoglycaemia can be reduced to below 12%, below 11%, below 10%, below 9%, below 8%, below 7%, below 6% or below 5% of diabetes type 2 patients receiving the combination of lixisenatide or/and a pharmaceutically acceptable salt thereof, basal insulin or/and a pharmaceutically acceptable salt thereof and optionally metformin or/and a pharmaceutically acceptable salt thereof, as described herein.

In the present invention, “symptomatic hypoglycaemia” is a condition associated with a clinical symptom that results from the hypoglycaemia, wherein the plasma glucose concentration is below 60 mg/dL (or below 3.3 mmol/L), below 50 mg/dL, or below 40 mg/dL. A clinical symptoms can be, for example, sweating, palpitations, hunger, restlessness, anxiety, fatigue, irritability, headache, loss of concentration, psychiatric disorders, visual disorders, transient sensory defects, transient motor defects, confusion, convulsions, and coma. In the present invention, one or more clinical symptoms of symptomatic hypoglycaemia, as indicated herein, can be selected.

Symptomatic hypoglycaemia may be associated with prompt recovery after oral carbohydrate administration.

In the present invention, “severe symptomatic hypoglycaemia” is a condition with a clinical symptom, as indicated herein, that results from hypoglycaemia, wherein the plasma glucose concentration is below 36 mg/dl (or below 2.0 mmol/L). Severe symptomatic hypoglycaemia can be associated with acute neurological impairment resulting from the hypoglycaemic event. In a severe symptomatic hypoglycaemia, the patient may require the assistance of another person, if, for example, the patient could not treat or help him/herself due to the acute neurological impairment. The definition of severe symptomatic hypoglycaemia may include all episodes in which neurological impairment is severe enough to prevent self-treatment and which were thus thought to place patients at risk for injury to themselves or others. The acute neurological impairment may be at least one selected from somnolence, psychiatric disorders, visual disorders, transient sensory defects, transient motor defects, confusion, convulsions, and coma.

Severe symptomatic hypoglycaemia may be associated with prompt recovery after oral carbohydrate, intravenous glucose, or/and glucagon administration.

A further aspect of the present invention is a method for preventing hypoglycemia in diabetes type 2 patients, said method comprising administering desPro³⁶Exendin-4(1-39)-Lys₆-NH₂ or/and a pharmaceutically acceptable salt thereof, in combination with a basal insulin or/and a pharmaceutically acceptable salt thereof, and optionally with metformin to a subject in need thereof. In particular, the combination as described herein may be administered. In the method of the present invention, the subject may be the subject defined herein.

Yet another aspect of the present invention refers to the use of the combination as described herein for the manufacture of a medicament for the treatment of a medical indication, as described herein. For example, the combination of the present invention can be used for the manufacture of a medicament for the treatment of diabetes type 2 patients, or for the treatment of conditions associated with diabetes type 2, such as improvement of glycemic control, reduction of the fasting plasma glucose concentration, for the improvement of glucose excursion, reduction of the postprandial plasma glucose concentration, improving the HbA1c value, or/and improvement of glucose tolerance. In another example, the combination as described herein can be used for the manufacture of a medicament for inducing weight loss in diabetes type 2 patients or/and for preventing weight gain in diabetes type 2 patients. In yet another example, the combination as described herein can be used for the manufacture of a medicament for preventing hypoglycaemia in diabetes type 2 patients. The medicament can be formulated as described herein. For example the medicament can comprise a parenteral formulation of AVE0010 or/and a pharmaceutically acceptable salt thereof, a parenteral formulation of the basal insulin or/and a pharmaceutically acceptable salt thereof, and an optional oral formulation of metformin or/and a pharmaceutically acceptable salt thereof.

The invention is further illustrated by the following example and figures.

### BRIEF DESCRIPTION OF DRAWINGS

FIG. 1—Study design

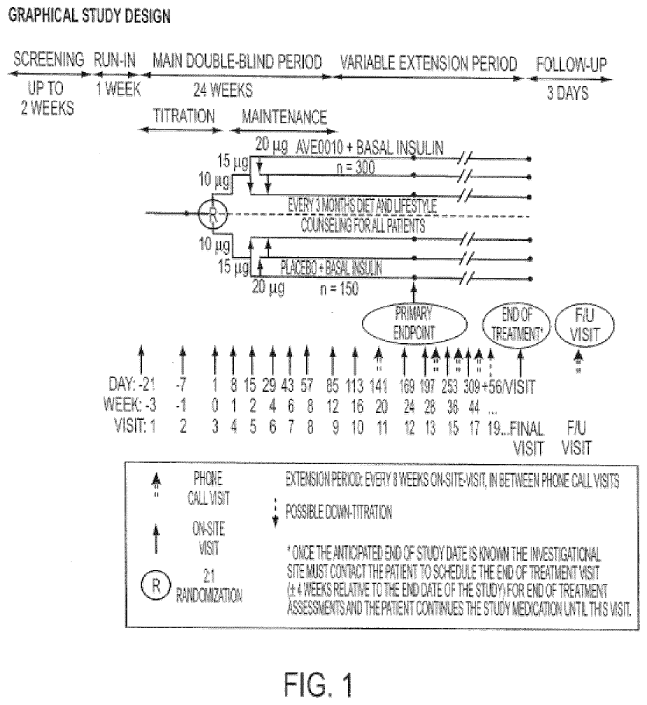

FIG. 2—Kaplan-Meier plot of time to treatment discontinuation due to any reason-Randomized population

FIG. 3—Plot of mean change in HbAlc (%) from baseline by visit up to Week 24 and at endpoint-mITT

FIG. 4—Plot of mean change in average 7-point Self Monitored Plasma Glucose (SMPG) (mmol/L) from baseline by visit up to Week 24 and at endpoint-mITT population

FIG. 5—Plot of mean change in fasting plasma glucose (mmol/L) from baseline by visit up to Week 24 and at endpoint-mITT population

FIG. 6—Plot of mean change in body weight (kg) from baseline by visit up to Week 24 and at endpoint-mITT population

FIG. 7—Plot of mean change in basal insulin dose (U) from baseline by visit up to Week 24 and at endpoint-mITT population

FIG. 8—Plot of mean change in HbA_{1c} (%) from baseline by visit and at endpoint-mITT population

FIG. 9—Plot of mean change in 2-hour postprandial plasma glucose (mmol/L) from baseline by visit and at endpoint-mITT population

FIG. 10—Plot of mean change in average 7-point Self Monitored Plasma Glucose (SMPG) (mmol/L) from baseline by visit and at endpoint-mITT population

FIG. 11—Plot of mean change in fasting plasma glucose (mmol/L) from baseline by visit and at endpoint-mITT population

FIG. 12—Plot of mean change in body weight (kg) from baseline by visit and at endpoint-mITT population

FIG. 13—Plot of mean change in basal insulin dose (U) from baseline by visit and at endpoint-mITT population

### EXAMPLE

The Example refers to a randomized, double-blind, placebo-controlled, 2-arm, parallel-group, multinational study assessing the efficacy and safety of lixisenatide in comparison to placebo as an add-on treatment to basal insulin in combination with or without metformin in patients with type 2 diabetes.

The Example refers to a randomized, double-blind, placebo-controlled. 2-arm. parallel-group, multinational study assessing the efficacy and safety of lixisenatide in comparison to Placebo as an add-on treatment to basal insulin in combination with or without metformin in patients with type 2 diabetes. The approximate minimum study duration per patient was 79 weeks (up to 3 weeks screening+24-week main treatment+variable extension+3 days follow-up). The study was conducted in 111 centers in 15 countries. The primary objective of the study was to assess the efficacy of lixisenatide on glycemic control in comparison to placebo in terms of HbA1c reduction (absolute change) over a period of 24 weeks.

A total of 496 patients were randomized to one of the two treatment groups (329 in the lixisenatide group and 167 in the placebo group) and 495 randomized patients were exposed to the investigational product (IP). Demographics and baseline characteristics were generally similar across the treatment groups. Four patients (2 on lixisenatide and 2 on placebo) were excluded from the mITT population for efficacy analyses due to lack of post-baseline efficacy data. During the whole-study treatment period. 115 (35.0%) lixisenatide-treated patients prematurely discontinued the IP, while 52 (31.1%) placebo-treated patients discontinued the IP. For both treatment groups, the main reason for treatment discontinuation was “other reasons” (15.8% for lixisenatide versus 13.2% for placebo) followed by “adverse events” (11.2% for lixisenatide versus 7.2% for placebo).

Efficacy analyses are based on the 24-week treatment: The least squared (LS) mean changes from baseline to Week 24 in HbA_{1c} were −0.74% for the lixisenatide group and −0.38% for the placebo group (LS mean difference vs. placebo=−0.36%: p-value=0.0002). A total of 86 patients (28.3%) in the lixisenatide group achieved HbA_{1c}<7% at Week 24 compared to 19 patients (12.0%) in the placebo group, and 44 (14.5%) lixisenatide-treated patients had HBA_{1c} ≤6.5% compared to 6 (3.8%) of placebo-treated patients. The HbA_{1c} responder analysis (HbA_{1c}≤6.5 or <7% at Week 24) using Cochran-Mantel-Haenszel (CMH) method showed a significant treatment difference versus placebo for lixisenatide group at Week 24 (p-value=0.0003 and p-value<0.0001, respectively).

Treatment with lixisenatide also improved post-prandial glycemic control as shown by the results for the 2-hour post-prandial plasma glucose (PPG) and glucose excursion assessment. A statistically significant improvement in PPG after a test meal was demonstrated in the lixisenatide group, compared with the placebo group with a LS mean difference of −3.81 mmol/L (p-value<0.0001). Furthermore, treatment with lixisenatide demonstrated a statistically significant improvement in the average of the 7-point self-monitored plasma glucose (SMPG) profile (LS mean difference of −0.88 mmol/L: p-value<0.0001) compared with the placebo group. For fasting plasma glucose, no statistically significant difference was observed between the treatment groups (LS mean difference versus placebo=−0.08 mmol/L; p-value=0.7579). Patients treated with lixisenatide showed a statistically significant decrease in body weight (LS mean difference of −1.28 kg: p-value<0.0001) compared with the placebo group without an adjustment for multiplicity. A total of 30 patients (12 [7.3%] in the placebo group and 18 [5.5%] in the lixisenatide group) received a rescue therapy. While achieving a better glycemic control, patients treated with lixisenatide also showed a statistically significant decrease in daily basal insulin dose compared to the placebo-treated patients. (LS mean difference of −3.69U. p-value=0.0120).

Safety analyses are based on the whole study treatment: Lixisenatide was well tolerated. The proportions of the patients with treatment emergent adverse events (TEAEs) were generally comparable between the two treatment groups (87.5% in the lixisenatide group versus 85.6% in the placebo group). Two patients in the lixisenatide group and two patients in the placebo group had TEAEs leading to death. The number of patients with serious TEAEs was 46 (14.0%) in the lixisenatide group and 17 (10.2%) in the placebo group. One hundred thirty-eight (42.1%) lixisenatide-treated patients had symptomatic hypoglycemic events as defined in the protocol during the on-treatment period. whereas 65 (38.9%) patients in the placebo group reported symptomatic hypoglycemia. Aside from hypoglycemia, the most frequently reported TEAE was nausea (29.3%) for the lixisenatide group and nasopharyngitis (12.6%) for the placebo group. Seven patients in the lixisenatide group (2.1%) and 1 patient in the placebo group (0.6%) experienced severe symptomatic hypoglycemia per the protocol definition. A total of 11 patients (8 [2.4%] lixisenatide-treated patients and 3 [1.8%] placebo-treated patients) reported 11 TEAEs adjudicated as an allergic reaction by the Allergic Reaction Assessment Committee (ARAC), and three of these events (2 events of anaphylactic reaction in the lixisenatide group and 1 angioedema in the placebo group) were adjudicated as possibly related to the IP. One lixisenatide-treated patient reported an event of pancreatitis, which was assessed as a recurrent pancreatitis and not related to the IP per the investigator.

### 1 OBJECTIVES

### 1.1 Primary Objective

The primary objective of this study was to assess the efficacy of lixisenatide on glycemic control in comparison to placebo as an add-on treatment to basal insulin in Type 2 Diabetes patients treated with basal insulin in terms of absolute HbA1c reduction over a period of 24 weeks.

### 1.2 Secondary Objective(s)

The secondary objectives of this study were:

To assess the effects of lixisenatide on:
Body weight,
2-hour postprandial plasma glucose after standardized meal challenge test,
Percentage of patients reaching HbA1c<7%,
Percentage of patients reaching HbA1c≤6.5%,
Fasting Plasma Glucose (FPG),
Change in 7-point Self Monitored Plasma Glucose (SMPG) profiles,
Change in basal insulin and total insulin doses.

To assess lixisenatide safety and tolerability.

To assess lixisenatide PK.

To assess anti-lixisenatide antibody development.

### 2 TRIAL DESIGN

This was a double-blind, randomized, placebo-controlled, 2-arm, parallel-group multinational study with an unbalanced 2:1 randomization ratio. The study was double-blind with regard to active and placebo treatments. The study drug volume (i.e., dose of active drug or matching placebo) was not blinded.

The patients were stratified by screening values of glycosylated hemoglobin A₁_{^{c}} (HbA₁_{^{c}}) (<8%, ≥8%) and metformin use at screening (Yes, No). After a screening period, patients were centrally randomized via interactive voice response system (IVRS) in a 2:1 ratio to either lixisenatide or placebo.

The approximate minimum study duration per patient was 79 weeks (up to 3 weeks screening+24 weeks main double-blind treatment+variable extension+3 days follow-up). Patients who completed the 24-week main double-blind period underwent a variable double-blind extension period, which ended for all patients approximately at the scheduled date of week 76 visit (V25) for the last randomized patient.

Patients who prematurely discontinued the IP were continued in the study up to the scheduled date of study completion. They were followed up according to the study procedures as specified in the protocol amendment (except 3-day safety post-treatment follow-up, pharmacokinetics assessment, and meal challenge test).

### 3 PRIMARY AND KEY SECONDARY ENDPOINTS

### 3.1 Primary Endpoint

The primary efficacy variable was the absolute change in HbA_{1c} from baseline to Week 24, which was defined as: HbA_{1c} at Week 24—HbA_{1c} at baseline.

If a patient discontinued the treatment prematurely or received rescue therapy during the main 24-week double-blind treatment period or did not have HbA_{1c} value at Week 24 visit, the last post-baseline HbA_{1c} measurement during the main 24-week double-blind on-treatment period was used as HbA_{1c} value at Week 24 (Last Observation Carried Forward [LOCF] procedure).

### 3.2 Secondary Endpoints

### 3.2.1 Efficacy Endpoints

For secondary efficacy variables, the same procedure for handling missing assessment/early discontinuation was applied as for the primary variable.

### Continuous Variables

Change in 2-hour postprandial plasma glucose (mmol/L) after a standardized meal from baseline to Week 24
Change in 7-point SMPG profiles (mmol/L) (ie, the average and each time point of the 7 points) from baseline to Week 24;
Change in FPG (mmol/L) from baseline to Week 24
Change in body weight (kg) from baseline to Week 24
Change in glucose excursion (2-hour postprandial plasma glucose-plasma glucose 30 minutes prior to the meal test before study drug administration) (mmol/L) after standardized meal challenge test from baseline to Week 24;
Change in daily basal insulin dose (U) and total insulin dose (U) from baseline to Week 24.

### Categorical Variables

Percentage of patients with HbA1c <7% at Week 24.
Percentage of patients with HbA1c ≤6.5% at Week 24.
Percentage of patients requiring rescue therapy during the main 24-week double-blind treatment period.
Percentage of patients with ≥5% weight loss (kg) from baseline to Week 24

### 3.2.2 Safety Endpoints

The safety analysis was based on the reported TEAEs and other safety information including symptomatic hypoglycemia and severe symptomatic hypoglycemia, local tolerability at injection site, allergic events (as adjudicated by ARAC), suspected pancreatitis, increased calcitonin, vital signs, 12-lead ECG and laboratory tests.

Major cardiovascular events were also collected and sent for adjudication by a Cardiovascular Adjudication Committee (CAC). The adjudicated and confirmed events by CAC from this study and other lixisenatide phase 3 studies will be pooled as necessary for analyses and summarized in a separate report based on the statistical analysis plan for the overall cardiovascular assessment of lixisenatide. The KRM/CSR will not present the summary of the adjudicated and confirmed CV events from this study.

### 4 SAMPLE SIZE CALCULATION ASSUMPTIONS

The sample size/power calculations were performed based on the primary-variable, change from baseline to Week 24 in HbA1c.

Three hundred patients in the lixisenatide treatment and 150 in the placebo treatment arm were expected to provide a power of 96% (or 86%) to detect differences of 0.5% (or 0.4%) in the change from baseline to Week 24 in HbA_{1c} between lixisenatide and placebo, assuming the common standard deviation (SD) was 1.3% with a 2-sided test at the 5% significance level.

### 5 STATISTICAL METHODS

### 5.1 Analysis Populations

The mITT population consists of all patients who were randomized, received at least one dose of double-blind IP, and had both a baseline assessment and at least one post-baseline assessment of any primary or secondary efficacy variables, irrespective of compliance with the study protocol and procedures.

The safety population was defined as all randomized patients who took at least one dose of the double-blind IP.

### 5.2 Primary Efficacy Analysis

The primary efficacy variable (change in HbA_{1c} from baseline to Week 24) was analyzed using an analysis of covariance (ANCOVA) model with treatment, randomization strata of screening HbA1c (<8.0, ≥8.00%). randomization strata of metformin use at screening (Yes, No) and country as fixed effects and using the baseline value as a covariate. Difference between lixisenatide and placebo and two-sided 95% confidence interval as wells as p-value were estimated within the framework of ANCOVA.

The LOCF procedure was used by taking the last available post-baseline on-treatment HbA_{1c} measurement (before the initiation of the new medication in the event of rescue therapy) as the HbA_{1c} value at Week 24.

The primary analysis of the primary efficacy variable \Vas performed based on the mITT population and the measurements obtained during the main 24-week double-bind on-treatment period for efficacy variables. The main 24-week double-blind on-treatment period for efficacy variables except those from the meal challenge test, 7-point SMPG, basal insulin dose and total insulin was defined as the time from the first dose of the double-blind IP up to 3 days (except for FPG by central laboratory, which was up to 1 day) after the last dose of the double-blind IP injection on or before V12/Week 24 visit (or D169 if V12/Week 24 visit was missing), or up to the introduction of the rescue therapy, whichever was the earliest. The main 24-week double-blind on-treatment period for efficacy variables from the meal challenge test including 2-hour PPG and glucose excursion, 7-point SMPG, basal insulin dose and total insulin was defined as the time from the first dose of the double-blind IP up to the date of the last dose of the double-blind IP injection on or before V12/Week 24 visit (or D169 if V12/Week 24 visit was missing), or up to the introduction of the rescue therapy, whichever was the earliest.

### 5.3 Secondary Efficacy Analysis

Once the primary variable was statistically significant at α=0.05, the testing procedure was performed to test the following secondary efficacy variables by the following prioritized order. The tests stop as soon as an endpoint was found not statistically significant at α=0.05.
1. Change in 2-hourpostprandial plasma glucose (mmol/L) after a standardized meal test from baseline to Week 24,
2. Change in the average of the 7-point SMPG from baseline to Week 24,
3. Change in FPG (mmol/L) from baseline to Week 24,
4. Change in body weight (kg) from baseline to Week 24,
5. Percentage of patients requiring rescue therapy during the main 24-week double-blind treatment period.

No multiplicity adjustment will be made on the other secondary efficacy variables, which are not mentioned above.

All continuous secondary efficacy variables at Week 24 as described in Section 3.2.1 were analyzed using the similar approach and ANCOVA model as described above for the primary analysis of the primary efficacy endpoint. The estimates of the treatment mean difference between lixisenatide and placebo and two-sided 95% confidence intervals were provided.

The following categorical secondary efficacy variables at Week 24 were analyzed using a Cochran-Mantel-Haenszel (CMH) method stratified on randomization strata (screening HbA_{1c} [<8.0, ≥8%] and metformin use at screening [Yes, No]):
Percentage of patients with HbA_{1c}<7.0% at Week 24,
Percentage of patients with HbA_{1c≤}6.5% at Week 24,
Percentage of patients requiring rescue therapy during the main 24-week double-blind treatment period.

Number and percentage of patients with ≥5% weight loss from baseline at Week 24 are presented by treatment groups.

All secondary endpoints at the end of treatment were only evaluated by descriptive statistics (mean, standard deviation, median and ranges provided in CSR).

### 5.4 Safety Analysis

The safety analyses were primarily based on the on-treatment period for the whole study. The on-treatment period for the whole study was defined as the time from the first dose of double-blind IP up to 3 days after the last dose of IP administration during the whole study period regardless of rescue status. The 3-day interval was chosen based on the half-life of the IP (approximately 5 times the half-life).

In addition, the safety analyses for the 24-week double-blind treatment period will be summarized in CSR.

The summary of safety results (descriptive statistics or frequency tables) is presented by treatment groups.

### 6 RESULTS

### 6.1 Study Patients

### 6.1.1 Patient Accountability

The study was conducted in 111 centers in 15 countries (Brazil, Canada, Chile, Egypt, France, Germany, India, Italy, Korea, Mexico, Puerto Rico, Russian Federation, Turkey, United Kingdom and United States). A total of 879 patients were screened and 496 were randomized to one of the two treatment groups. The main reason for screening failure was HbA_{1c} value at the screening visit was out of the protocol defined range (205 [23.3%] out of 879 screened patients).

Of the 496 randomized patients, 495 were exposed to the IP. One patient from lixisenatide group was not exposed to the IP. Four patients (2 in the lixisenatide group and 2 in the placebo group) were excluded from mITT population for efficacy analyses due to lack of post-baseline efficacy data. Table 1 provides the number of patients included in each analysis population.

TABLE 1
Analysis populations-Randomized population

| | Placebo | Lixisenatide | All |
|---|---|---|---|
| Randomized population | 167 (100%) | 329 (100%) | 496 (100%) |
| Efficacy population | 165 (98.8%) | 326 (99.1%) | 491 (99.0%) |
| Modified Intent-to-Treat | | | |
| (mITT) | | | |
| Safety population | 167 (100%) | 328 (99.1%) | 495 (99.8%) |

Note:
The Safety patients are tabulated according to treatment actually received (as treated).
For the efficacy populations, patients are tabulated according to their randomized treatment (as randomized).

### 6.1.2 Study Disposition

Table 2 provides the summary of patient disposition for each treatment group. During the whole-study treatment period, 115 (35.0%) lixisenatide-treated patients prematurely discontinued the IP, while 52 (31.1%) placebo-treated patients discontinued the IP. For both treatment groups, the main reason for treatment discontinuation was “other reasons” (52 patients [15.8%] for lixisenatide and 22 patients [13.2%] for placebo), mostly being personal reasons such as moving out of town, lost job, family issues or too busy to commit to the visit schedules, followed by “adverse events” (37 patients [11.2%] including 2 non-TEAEs for lixisenatide versus 12 patients [7.2%] for placebo).

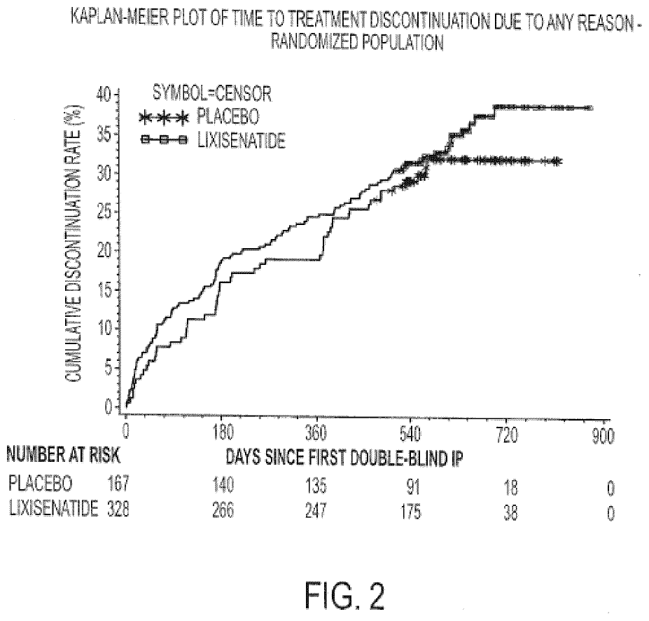

For the 24-week main treatment period, 53 (16.1%) patients in lixisenatide group and 20 (12.0%) in placebo prematurely discontinued the IP with the main reason being adverse events (26 patients, 7.9%) for lixisenatide group and “other” (8 patients, 4.8%) for placebo group. The time-to-onset of treatment discontinuation due to any reason for the overall treatment period is depicted in FIG. 2. A higher discontinuation rate was observed for the lixisenatide group.

TABLE 2
Patient disposition-Randomized population

| | Placebo | | Lixisenatide | |
|---|---|---|---|---|
| | (N = 167) | | (N = 329) | |
| Randomized and treated- | 167 | (100%) | 328 | (99.7%) |
| Did not complete 24-week double- | 20 | (12.0%) | 53 | (16.1%) |
| blind study treatment | | | | |
| Subject's request for 24-week | 15 | (9.0%) | 46 | (14.0%) |
| treatment discontinuation | | | | |
| Reason for 24-week treatment | 20 | (12.0%) | 53 | (16.1%) |
| discontinuation | | | | |
| Adverse event | 4 | (2.4%) | 26 | (7.9%) |
| Lack of efficacy | 3 | (1.8%) | 3 | (0.9%) |
| Poor compliance to protocol | 4 | (2.4%) | 6 | (1.8%) |
| Lost to follow-up | 1 | (0.6%) | 0 | |
| Other | 8 | (4.8%) | 18 | (5.5%) |
| Did not complete double-blind study | 52 | (31.1%) | 115 | (35.0%) |
| treatment | | | | |
| Subject's request for treatment | 41 | (24.6%) | 93 | (28.3%) |
| discontinuation | | | | |
| Reason for study treatment | 52 | (31.1%) | 115 | (35.0%) |
| discontinuation | | | | |
| Adverse event | 12 | (7.2%) | 37 | (11.2%) |
| Lack of efficacy | 11 | (6.6%) | 11 | (3.3%) |
| Poor compliance to protocol | 6 | (3.6%) | 13 | (4.0%) |
| Lost to follow-up | 1 | (0.6%) | 2 | (0.6%) |
| Other | 22 | (13.2%) | 52 | (15.8%) |
| Status at last study contact | 167 | (100%) | 329 | (100%) |
| Alive | 163 | (97.6%) | 320 | (97.3%) |
| Lost to follow-up | 2 | (1.2%) | 6 | (1.8%) |
| Dead | 2 | (1.2%) | 3 | (0.9%) |

Note:
Percentages are calculated using the number of randomized patients as denominator

### 6.1.3 Demographics and Baseline Characteristics

The demographic and patient baseline characteristics were generally similar between the two treatment groups for the safety population (Table 3). The median age of the study population was 58.0 years. The majority of the patients were Caucasian (77.6%). The lixisenatide group had more female patients in percentage (55.5% female and 44.5% male) than the placebo group (50.9% female and 49.1% male).

TABLE 3
Demographics and patient characteristics at
screening or baseline-Safety population

| | Placebo | | Lixisenatide | | All | |
|---|---|---|---|---|---|---|
| | (N = 167) | | (N = 328) | | (N = 495) | |
| Age (years) | | | | | | |
| Number | 167 | | 328 | | 495 | |
| Mean (SD) | 56.9 | (9.8) | 57.4 | (9.5) | 57.2 | (9.6) |
| Median | 57.0 | | 58.0 | | 58.0 | |
| Min:Max | 29:81 | | 34:80 | | 29:81 | |
| Age Group (years) [n (%)] | | | | | | |
| Number | 167 | | 328 | | 495 | |
| <50 | 38 | (22.8%) | 64 | (19.5%) | 102 | (20.6%) |
| ≥50 to <65 | 93 | (55.7%) | 194 | (59.1%) | 287 | (58.0%) |
| ≥65 to <75 | 32 | (19.2%) | 61 | (18.6%) | 93 | (18.8%) |
| ≥75 | 4 | (2.4%) | 9 | (2.7%) | 13 | (2.6%) |
| Sex [n (%)] | | | | | | |
| Number | 167 | | 328 | | 495 | |
| Male | 82 | (49.1%) | 146 | (44.5%) | 228 | (46.1%) |
| Female | 85 | (50.9%) | 182 | (55.5%) | 267 | (53.9%) |
| Race [n (%)] | | | | | | |
| Number | 167 | | 328 | | 495 | |
| Caucasian/White | 130 | (77.8%) | 254 | (77.4%) | 384 | (77.6%) |
| Black | 6 | (3.6%) | 14 | (4.3%) | 20 | (4.0%) |
| Asian/Oriental | 30 | (18.0%) | 53 | (16.2%) | 83 | (16.8%) |
| Other | 1 | (0.6%) | 7 | (2.1%) | 8 | (1.6%) |
| Ethnicity [n (%)] | | | | | | |
| Number | 167 | | 328 | | 495 | |
| Hispanic | 40 | (24.0%) | 94 | (28.7%) | 134 | (27.1%) |
| Non Hispanic | 127 | (76.0%) | 234 | (71.3%) | 361 | (72.9%) |
| Screening HbA1c (%) | | | | | | |
| Number | 167 | | 328 | | 495 | |
| Mean (SD) | 8.46 | (0.81) | 8.49 | (0.83) | 8.48 | (0.82) |
| Median | 8.50 | | 8.50 | | 8.50 | |
| Min:Max | 7.0:10.0 | | 7.0:10.0 | | 7.0:10.0 | |
| Randomization strata of screening | | | | | | |
| HbA1c (%) [n (%)] | | | | | | |
| Number | 167 | | 328 | | 495 | |
| <8 | 51 | (30.5%) | 98 | (29.9%) | 149 | (30.1%) |
| ≥8 | 116 | (69.5%) | 230 | (70.1%) | 346 | (69.9%) |
| Randomization strata of metformin | | | | | | |
| use at screening [n (%)] | | | | | | |
| Number | 167 | | 328 | | 495 | |
| Yes | 131 | (78.4%) | 259 | (79.0%) | 390 | (78.8%) |
| No | 36 | (21.6%) | 69 | (21.0%) | 105 | (21.2%) |
| Baseline BMI (kg/m²) | | | | | | |
| Number | 167 | | 328 | | 495 | |
| Mean (SD) | 32.56 | (6.32) | 31.91 | (6.17) | 32.13 | (6.22) |
| Median | 32.33 | | 31.23 | | 31.35 | |
| Min:Max | 20.1:58.4 | | 20.7:64.4 | | 20.1:64.4 | |
| Baseline BMI Categories (kg/m²) | | | | | | |
| [n (%)] | | | | | | |
| Number | 167 | | 328 | | 495 | |
| <30 | 61 | (36.5%) | 137 | (41.8%) | 198 | (40.0%) |
| ≥30 | 106 | (63.5%) | 191 | (58.2%) | 297 | (60.0%) |

BMI = Body Mass Index.

Disease characteristics including diabetic history were generally comparable between the two treatment groups (Table 4). One placebo-treated patient (#840608010) had Type 1 diabetes and was discontinued from the study shortly after the identification of the diagnosis.

The mean duration of basal insulin treatment for the study population was 3.11 years (Table 5). Majority of the patients took either long-acting insulin analogues (glargine 50.1%, detemir 8.7%) or NPH (40.0%) during screening and continued into the study treatment period with a few exceptions (1.6%) who took pre-mixed insulin instead. Eight patients (5 on lixisenatide and 3 on placebo) took pre-mixed insulin at screening and continued into the study. Two placebo-treated patients took two types of insulin (one on detemir+glargine and another on NPH+Novolin® 70/30 mix) during screening and continued into the study. All insulin usage including pre-mixed insulin is displayed in Table 5.

Three hundreds and ninety-two patients (79.2%) were on metformin at the screening visit with a similar usage proportion in the two treatment groups (lixisenatide 79.6% and placebo 78.4%, (Table 6). There were two discrepancies in the number of patients between “randomization strata of metformin use at screening” and actual “metformin use at screening” due to randomization strata errors.

TABLE 4
Disease characteristics at screening or baseline-Safety population

| | Placebo | | Lixisenatide | | All | |
|---|---|---|---|---|---|---|
| | (N = 167) | | (N = 328) | | (N = 495) | |
| Duration of diabetes (years) | | | | | | |
| Number | 167 | | 328 | | 495 | |
| Mean (SD) | 12.43 | (6.33) | 12.48 | (7.04) | 12.46 | (6.80) |
| Median | 11.39 | | 11.43 | | 11.39 | |
| Min:Max | 2.3:34.8 | | 1.0:41.6 | | 1.0:41.6 | |
| Age at onset of Type 2 diabetes | | | | | | |
| (years) | | | | | | |
| Number | 167 | | 328 | | 495 | |
| Mean (SD) | 44.55 | (9.16) | 44.85 | (9.08) | 44.75 | (9.10) |
| Median | 45.22 | | 45.00 | | 45.00 | |
| Min:Max | 15.0:72.0 | | 18.0:68.0 | | 15.0:72.0 | |
| History of gestational diabetes | | | | | | |
| [n (%)] | | | | | | |
| Number (Female) | 85 | | 182 | | 267 | |
| Yes (Female) | 6 | (7.1%) | 19 | (10.4%) | 25 | (9.4%) |
| No (Female) | 79 | (92.9%) | 163 | (89.6%) | 242 | (90.6%) |
| Prior use of GLP-1 receptor | | | | | | |
| agonist [n (%)] | | | | | | |
| Number | 167 | | 328 | | 495 | |
| Yes | 6 | (3.6%) | 7 | (2.1%) | 13 | (2.6%) |
| No | 161 | (96.4%) | 321 | (97.9%) | 482 | (97.4%) |
| Diabetic retinopathy [n (%)] | | | | | | |
| Number | 167 | | 328 | | 495 | |
| Yes | 33 | (19.8%) | 66 | (20.1%) | 99 | (20.0%) |
| No | 132 | (79.0%) | 251 | (76.5%) | 383 | (77.4%) |
| Unknown | 2 | (1.2%) | 11 | (3.4%) | 13 | (2.6%) |
| Diabetic sensory or motor | | | | | | |
| neuropathy [n (%)] | | | | | | |
| Number | 167 | | 328 | | 495 | |
| Yes | 50 | (29.9%) | 99 | (30.2%) | 149 | (30.1%) |
| No | 116 | (69.5%) | 225 | (68.6%) | 341 | (68.9%) |
| Unknown | 1 | (0.6%) | 4 | (1.2%) | 5 | (1.0%) |
| Diabetic autonomic neuropathy | | | | | | |
| [n (%)] | | | | | | |
| Number | 167 | | 328 | | 495 | |
| Yes | 9 | (5.4%) | 8 | (2.4%) | 17 | (3.4%) |
| No | 156 | (93.4%) | 314 | (95.7%) | 470 | (94.9%) |
| Unknown | 2 | (1.2%) | 6 | (1.8%) | 8 | (1.6%) |
| Diabetic nephropathy [n (%)] | | | | | | |
| Number | 167 | | 328 | | 495 | |
| Yes | 15 | (9.0%) | 31 | (9.5%) | 46 | (9.3%) |
| Microalbuminuria | 6 | (3.6%) | 21 | (6.4%) | 27 | (5.5%) |
| Overt proteinuria | 2 | (1.2%) | 1 | (0.3%) | 3 | (0.6%) |
| Impaired renal function | 3 | (1.8%) | 1 | (0.3%) | 4 | (0.8%) |
| Dialysis or transplantation | 0 | | 0 | | 0 | |
| No | 150 | (89.8%) | 291 | (88.7%) | 441 | (89.1%) |
| Unknown | 2 | (1.2%) | 6 | (1.8%) | 8 | (1.6%) |
| Categorized albuminuria at | | | | | | |
| randomization [n (%)] | | | | | | |
| Number | 36 | | 56 | | 92 | |
| <3 mg/L (Not reportable) | 3 | (8.3%) | 5 | (8.9%) | 8 | (8.7%) |
| ≥3 mg/L (Reportable) | 33 | (91.7%) | 51 | (91.1%) | 84 | (91.3%) |
| <20 mg/L | 17 | (47.2%) | 26 | (46.4%) | 43 | (46.7%) |
| ≥20-<200 mg/L | 10 | (27.8%) | 19 | (33.9%) | 29 | (31.5%) |
| ≥200 mg/L | 6 | (16.7%) | 6 | (10.7%) | 12 | (13.0%) |
| Creatinine clearance at screening | | | | | | |
| (ml/min) | | | | | | |
| Number | 167 | | 326 | | 493 | |
| Mean (SD) | 119.16 | (45.81) | 118.05 | (45.05) | 118.43 | (45.27) |
| Median | 108.66 | | 109.13 | | 109.03 | |
| Min:Max | 22.8:329.6 | | 32.4:358.2 | | 22.8:358.2 | |
| Creatinine clearance categories | | | | | | |
| at screening [n (%)] | | | | | | |
| Number | 167 | | 326 | | 493 | |
| <30 ml/min (severe renal | 1 | (0.6%) | 0 | | 1 | (0.2%) |
| impairment) | | | | | | |
| ≥30-<50 ml/min (moderate | 2 | (1.2%) | 4 | (1.2%) | 6 | (1.2%) |
| renal impairment) | | | | | | |
| ≥50-<80 ml/min (mild renal | 22 | (13.2%) | 51 | (15.6%) | 73 | (14.8%) |
| impairment) | | | | | | |
| >80 ml/min (no renal | 142 | (85.0%) | 271 | (83.1%) | 413 | (83.8%) |
| impairment) | | | | | | |

GLP-1 = Glucagon like peptide-1.
Creatinine clearance value is derived using the equation of Cockroft and Gault.

TABLE 5
Diabetes history-Basal insulin at screening-Safety population

| | Placebo | | Lixisenatide | | All | |
|---|---|---|---|---|---|---|
| | (N = 167) | | (N = 328) | | (N = 495) | |
| Duration of treatment with basal | | | | | | |
| insulin (years) | | | | | | |
| Number | 167 | | 328 | | 495 | |
| Mean (SD) | 3.20 | (3.96) | 3.06 | (3.37) | 3.11 | (3.57) |
| Median | 1.67 | | 1.80 | | 1.75 | |
| Min:Max | 0.2:21.4 | | 0.2:20.4 | | 0.2:21.4 | |
| Insulin at screening | | | | | | |
| Number | 167 | | 328 | | 495 | |
| Glargine | 83 | (49.7%) | 165 | (50.3%) | 248 | (50.1%) |
| Detemir | 19 | (11.4%) | 24 | (7.3%) | 43 | (8.7%) |
| NPH¹ | 64 | (38.3%) | 134 | (40.9%) | 198 | (40.0%) |
| Lente | 0 | | 0 | | 0 | |
| Ultralente | 0 | | 0 | | 0 | |
| Premix(Mixed insulin)^{2,3} | 3 | (1.8%) | 5 | (1.5%) | 8 | (1.6%) |
| Regimen at screening | | | | | | |
| Number | 167 | | 328 | | 495 | |
| Morning | 25 | (15.0%) | 54 | (16.5%) | 79 | (16.0%) |
| Evening | 67 | (40.1%) | 137 | (41.8%) | 204 | (41.2%) |
| Morning and evening | 75 | (44.9%) | 137 | (41.8%) | 212 | (42.8%) |
| Daily dose at screening | | | | | | |
| Total | | | | | | |
| Number | 167 | | 328 | | 495 | |
| Mean (SD) | 57.55 | (34.72) | 53.95 | (34.08) | 55.16 | (34.31) |
| Median | 46.00 | | 42.00 | | 44.00 | |
| Min:Max | 0.0:200.0 | | 0.0⁴:400.0 | | 0.0⁴:400.0 | |
| Glargine | | | | | | |
| Number | 83 | | 165 | | 248 | |
| Mean (SD) | 61.60 | (36.77) | 56.53 | (39.47) | 58.23 | (38.59) |
| Median | 48.00 | | 43.00 | | 45.00 | |
| Min:Max | 30.0:190.0 | | 0.0:400.0 | | 0.0:400.0 | |
| Detemir | | | | | | |
| Number | 19 | | 24 | | 43 | |
| Mean (SD) | 72.53 | (49.52) | 57.29 | (37.75) | 64.02 | (43.47) |
| Median | 60.00 | | 40.00 | | 44.00 | |
| Min:Max | 30.0:200.0 | | 25.0:150.0 | | 25.0:200.0 | |
| NPH¹ | | | | | | |
| Number | 64 | | 134 | | 198 | |
| Mean (SD) | 45.78 | (16.83) | 47.42 | (19.92) | 46.89 | (18.95) |
| Median | 40.00 | | 40.00 | | 40.00 | |
| Min:Max | 30.0:100.0 | | 20.0:140.0 | | 20.0:140.0 | |
| Premix(Mixed insulin)^{2,3} | | | | | | |
| Number | 3 | | 5 | | 8 | |
| Mean (SD) | 63.33 | (65.06) | 127.60 | (33.81) | 103.50 | (54.49) |

| | | | |
|---|---|---|---|
| Median | 60.00 | 110.00 | 106.00 |
| Min:Max | 0.0:130.0 | 100.0:176.0 | 0.0:176.0 |

¹NPH included Isophand insulin and Insulin human injection, isophane.
²Protocol deviation.
³Pre-mixed insulin included Novolin 70/30 mix and Humalog 75/25 mix.
⁴Two patients (840612006 lixisenatide and 630625001 placebo) did not take their basal insulin on the date of screening.

TABLE 6
Disease characteristics-Metformin at screening or baseline-Safety population

| | Placebo | | Lixisenatide | | All | |
|---|---|---|---|---|---|---|
| | (N = 167) | | (N = 328) | | (N = 495) | |
| Metformin use at screening [n (%)] | | | | | | |
| Number | 167 | | 328 | | 495 | |
| Yes | 131 | (78.4%) | 261 | (79.6%) | 392 | (79.2%) |
| No | 36 | (21.6%) | 67 | (20.4%) | 103 | (20.8%) |
| Duration of metformin | | | | | | |
| treatment (years) | | | | | | |
| Number | 131 | | 260 | | 391 | |
| Mean (SD) | 6.47 | (4.89) | 7.01 | (5.94) | 6.83 | (5.61) |
| Median | 4.84 | | 5.74 | | 5.40 | |
| Min:Max | 0.3:23.1 | | 0.1:29.5 | | 0.1:29.5 | |
| Daily dose of metformin | | | | | | |
| at baseline (mg) | | | | | | |
| Number | 131 | | 261 | | 392 | |
| Mean (SD) | 2008.02 | (441.88) | 1961.02 | (459.07) | 1976.72 | (453.38) |
| Median | 2000.00 | | 2000.00 | | 2000.00 | |
| Min:Max | 100.0:3000.0 | | 850.0:4200.0 | | 850.0:4200.0 | |
| Categorized daily dose of | | | | | | |
| metformin at baseline (mg) [n (%)] | | | | | | |
| Number | 131 | | 261 | | 392 | |
| <1500 | 2 | (1.5%) | 9 | (3.4%) | 11 | (2.8%) |
| ≥1500-<2500 | 98 | (74.8%) | 203 | (77.8%) | 301 | (76.8%) |
| ≥2500-<3000 | 22 | (16.8%) | 32 | (12.3%) | 54 | (13.8%) |
| ≥5000 | 9 | (6.9%) | 17 | (6.5%) | 26 | (6.6%) |

Basline efficacy variables including HbA_{1c} were generally comparable between two treatment groups for the safety population (Table 7).

TABLE 7
Baseline efficacy variables-Safety population

| | Placebo | | Lixisenatide | | All | |
|---|---|---|---|---|---|---|
| | (N = 167) | | (N = 328) | | (N = 495) | |
| HbA1c (%) | | | | | | |
| Number | 167 | | 3258 | | 495 | |
| Mean (SD) | 8.37 | (0.84) | 8.42 | (0.88) | 8.40 | (0.87) |
| Median | 8.40 | | 8.40 | | 8.40 | |
| Min:Max | 6.7:10.5 | | 6.0:10.8 | | 6.0:10.8 | |
| Weight (kg) | | | | | | |
| Number | 167 | | 328 | | 495 | |
| Mean (SD) | 88.94 | (20.84) | 87.10 | (20.01) | 87.72 | (20.29) |
| Median | 86.00 | | 84.95 | | 85.40 | |
| Min:Max | 50.2:160.5 | | 46.5:157.8 | | 46.5:160.5 | |
| FPG (mmol/L) | | | | | | |
| Number | 167 | | 328 | | 495 | |
| Mean (SD) | 8.05 | (2.65) | 8.13 | (2.83) | 8.10 | (2.76) |
| Median | 7.60 | | 7.90 | | 7.80 | |
| Min:Max | 2.6:15.5 | | 2.5:23.9 | | 2.5:23.9 | |
| 2-hour postprandial | | | | | | |
| plasma glucose | | | | | | |
| (mmol/L) | | | | | | |
| Number | 153 | | 302 | | 455 | |
| Mean (SD) | 16.11 | (3.86) | 16.47 | (4.30) | 16.35 | (4.15) |
| Median | 16.10 | | 16.40 | | 16.30 | |
| Min:Max | 4.5:27.1 | | 5.6:29.3 | | 4.5:29.3 | |
| Glucose excursion | | | | | | |
| (mmol/L) | | | | | | |
| Number | 153 | | 301 | | 454 | |
| Mean (SD) | 7.32 | (3.43) | 7.59 | (3.60) | 7.50 | (3.54) |
| Median | 7.20 | | 7.70 | | 7.50 | |
| Min:Max | −4.7:15.5 | | −1.9:17.8 | | −4.7:17.8 | |
| Average 7-point | | | | | | |
| SMPG (mmol/L) | | | | | | |
| Number | 155 | | 301 | | 456 | |
| Mean (SD) | 10.58 | (2.69) | 10.76 | (2.61) | 10.70 | (2.64) |
| Median | 10.16 | | 10.53 | | 10.45 | |
| Min:Max | 6.8:23.4 | | 5.3:21.4 | | 5.3:23.4 | |
| Basal insulin | | | | | | |
| dose (U) | | | | | | |
| Number | 167 | | 328 | | 495 | |
| Mean (SD) | 57.73 | (34.54) | 53.43 | (33.89) | 54.88 | (34.14) |
| Median | 46.00 | | 41.00 | | 42.00 | |
| Min:Max | 30.0:200.0 | | 20.0:400.0 | | 20.0:400.0 | |
| Total insulin | | | | | | |
| dose (U) | | | | | | |
| Number | 167 | | 328 | | 495 | |
| Mean (SD) | 57.73 | (34.54) | 53.43 | (33.89) | 54.88 | (34.14) |

| | | | |
|---|---|---|---|
| Median | 46.00 | 41.00 | 42.00 |
| Min:Max | 30.0:200.0 | 20.0:400.0 | 20.0:400.0 |

FPG = Fasting Plasma Glucose.
SMPG = Self-Monitored Blood Glucose.
Glucose excursion = 2-hour postprandial plasma glucose − plasma glucose 30 minutes prior to the meal test before study drug administration.

### 6.1.4 Dosage and Duration

The average treatment exposure was 491.5 days (70.2 weeks) for the lixisenatide group and 510.4 days (72.9 weeks) for the placebo-group (Table 8). Of the 495 patients. 270 (82.3%) patients in the lixisenatide group and 146 (87.4%) patients in the placebo group received at least 169 days (24 weeks) of treatment; moreover, 171 (52.1%) patients in the lixisenatide group and 89 (53.3%) patients in the placebo group had at least 547 days (18 months) of treatment. Six patients (3 for lixisenatide and 3 for placebo) had a missing last administration date; of them, three were lost to follow-up (2 for lixisenatide and 1 for placebo) and hence their treatment durations were set to missing following the SAP data handling convention.

For the lixisenatide group, 286 (87.2%) patients were at the target total daily dose of 20 μg both at the end of the 24-week double-blind treatment period and at the end of whole double-blind treatment (Tables 9 and 10). For the placebo group, 161 (96.4%) patients and 162 (97.0%) patients were at the target total daily dose of 20 μg at the end of 24-week double-blind treatment period and at the end of whole double-blind treatment, respectively (Tables 9 and 10).

TABLE 8
Exposure-Safety population

| | | Placebo | | Lixisenatide | |
|---|---|---|---|---|---|
| | | (N = 167) | | (N = 328) | |
| Cumulative duration of treatment | | 229.2 | | 437.4 | |
| exposure (patient years) | | | | | |
| Duration of study treatment (days) | | | | | |
| Number | | 164 | | 325 | |
| Mean (SD) | | 510.4 | (210.6) | 491.5 | (233.9) |
| Median | | 559.0 | | 560.0 | |
| Min:Max | | 1:817 | | 1:875 | |
| Duration of study treatment by | | | | | |
| category [n (%)] | | | | | |
| Missing duration | | 3 | (1.8%) | 3 | (0.9%) |
| 1-14 | days | 3 | (1.8%) | 12 | (3.7%) |
| 15-28 | days | 3 | (1.8%) | 9 | (2.7%) |
| 29-56 | days | 4 | (2.4%) | 9 | (2.7%) |
| 57-84 | days | 3 | (1.8%) | 9 | (2.7%) |
| 85-168 | days | 5 | (3.0%) | 16 | (4.9%) |
| 169-364 | days | 11 | (6.6%) | 25 | (7.6%) |
| 365-546 | days | 46 | (27.5%) | 74 | (22.6%) |
| 547-728 | days | 72 | (43.1%) | 137 | (41.8%) |
| >728 | days | 17 | (10.2%) | 34 | (10.4%) |
| Cumulative duration of study | | | | | |
| treatment by category [n (%)] | | | | | |
| Missing duration | | 3 | (1.8%) | 3 | (0.9%) |
| ≥1 | day | 164 | (98.2%) | 325 | (99.1%) |
| ≥15 | days | 161 | (96.4%) | 313 | (95.4%) |
| ≥29 | days | 158 | (94.6%) | 304 | (92.7%) |
| ≥57 | days | 154 | (92.2%) | 295 | (89.9%) |
| ≥85 | days | 151 | (90.4%) | 286 | (87.2%) |
| ≥169 | days | 146 | (87.4%) | 270 | (82.3%) |
| ≥365 | days | 135 | (80.8%) | 245 | (74.7%) |
| ≥547 | days | 89 | (53.3%) | 171 | (52.1%) |
| ≥729 | days | 17 | (10.2%) | 34 | (10.4%) |

Duration of exposure = (date of the last double-blind investigational product injection − date of the first double-blind investigational product injection) + 1.

TABLE 9
Number (%) of patients by dose at the
end of titration-Safety population

| Dose at the end | Placebo | | Lixisenatide | |
|---|---|---|---|---|
| of titration | (N = 167) | | (N = 328) | |
| 10 μg | 2 | (1.2%) | 16 | (4.9%) |
| 15 μg | 10 | (6.0%) | 31 | (9.5%) |
| 20 μg | 155 | (92.8%) | 281 | (85.7%) |

Dose = Dose of active drug or volume-matched placebo.
The scheduled visit for end of titration per protocol would be Visit 5/Week 2.
Note:
Percentages are calculated using the number of safety patients as the denominator.

TABLE 10
Number (%) of patients by final total daily dose at
the end of the 24-week treatment - Safety population

| Dose at the end | Placebo | Lixisenatide |
|---|---|---|
| of the 24-week | (N = 167) | (N = 328) |
| 10 μg | 2 (1.2%) | 25 (7.6%) |
| 15 μg | 4 (2.4%) | 17 (5.2%) |
| 20 μg | 161 (96.4%) | 286 (87.2%) |

Dose = Dose of active drug or volume-matched placebo.
Note:
Percentages are calculated using the number of safety patients as the denominator.

TABLE 11
Number (%) of patients by final total daily dose
at the end of the treatment - Safety population

| | Placebo | Lixisenatide |
|---|---|---|
| Final Dose | (N = 167) | (N = 328) |
| 10 μg | 2 (1.2%) | 23 (7.0%) |
| 15 μg | 3 (1.8%) | 19 (5.8%) |
| 20 μg | 162 (97.0%) | 286 (87.2%) |

Dose = Dose of active drug or volume-matched placebo.
Note:
Percentages are calculated using the number of safety patients as the denominator.

### 6.2 Efficacy

6.2.1 Primary efficacy endpoint

### Main Analysis

Table 11 summarizes the results of the primary efficacy parameter, change from baseline to Week 24 (LOCF) in HbA_{1c} using an ANCOVA analysis.

The pre-specified primary analysis showed that treatment with lixisenatide resulted in a statistically significant decrease in HbA_{1c} from Baseline to Week 24, compared with the placebo group (LS mean difference versus the placebo group=−0.36%; p-value=0.0002).

TABLE 12
Mean change in HbA_{1c} (%) from baseline
to Week 24 - mITT population

| HbA1c | Placebo | Lixisenatide |
|---|---|---|
| (%) | (N = 165) | (N = 326) |
| Baseline | | |
| Number | 158 | 304 |
| Mean (SD) | 8.38 (0.83) | 8.39 (0.86) |
| Median | 8.40 | 8.40 |
| Min:Max | 6.8:10.5 | 6.0:10.8 |
| Week 24 (LOCF) | | |
| Number | 158 | 304 |
| Mean (SD) | 8.13 (1.17) | 7.76 (1.18) |
| Median | 7.90 | 7.60 |
| Min:Max | 6.0:11.6 | 5.6:14.1 |
| Change from baseline to Week 24 | | |
| (LOCF) | | |
| Number | 158 | 304 |
| Mean (SD) | −0.24 (0.98) | −0.63 (1.08) |
| Median | −0.30 | −0.60 |
| Min:Max | −2.7:2.7 | −3.5:5.7 |
| LS Mean (SE) ^{(a)} | −0.38 (0.107) | −0.74 (0.090) |
| LS Mean difference (SE) vs. | — | −0.36 (0.096) |
| placebo ^{(a)} | | |
| 95% CI | — | (−0.550 to −0.174) |
| p-value | — | 0.0002 |

LOCF = Last observation carried forward.
^{(a)} Analysis of covariance (ANCOVA) model with treatment groups (lixisenatide and placebo), randomization strata of screening HbA1c (<8.0, ≥8.0%), randomization strata of metformin use at screening (Yes, No), country as fixed effects and baseline HbA1c value as a covariate.
The analysis included measurements obtained before the introduction of rescue medication and up to 3 days after the last dose of the double-blind investigational product injection on or before Visit 12 (Week 24), or Day 169 if Visit 12 (Week 24) is not available. Patients with both baseline and Week 24 (LOCF) measurements are included.

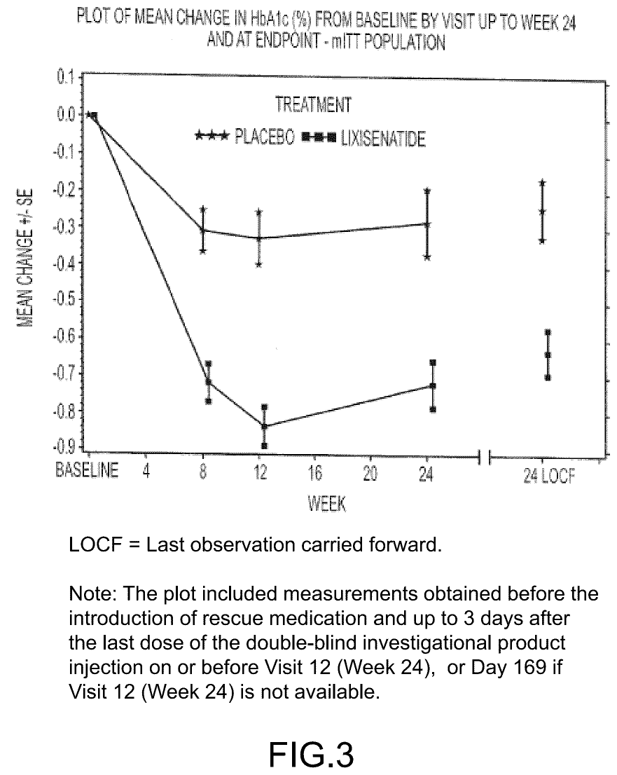

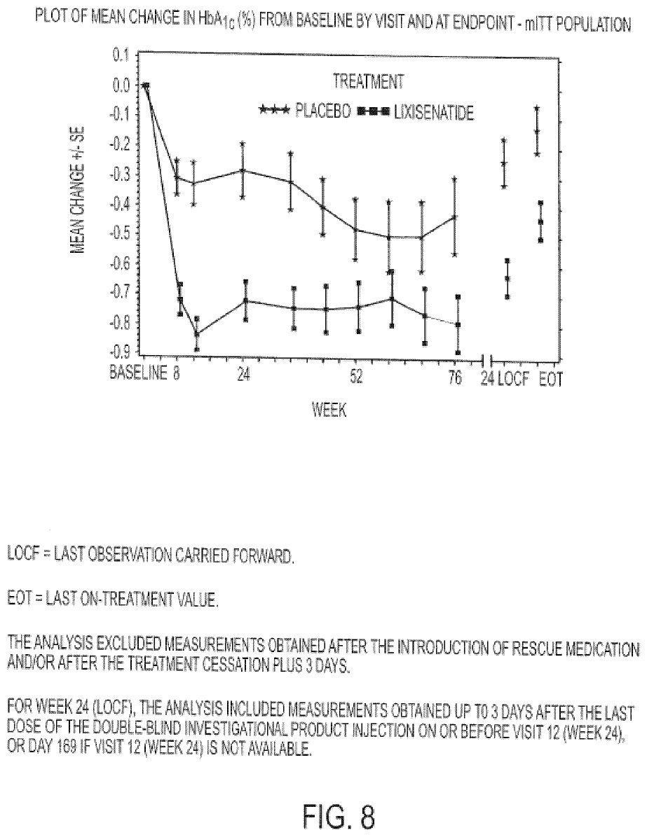

FIG. 3 illustrates the Mean (±SE) change from baseline in HbA_{1c} during the main 24-week double-blind treatment period. FIG. 8 in the appendix illustrates the Mean (±SE) change from baseline in HbA_{1c} over time up to Week 76. The HbA_{1c} reduction was relatively maintained over time beyond 24 weeks.

Table 13 summarizes-the proportion of patients with treatment response in HbA_{1c}56.5% or <7% at Week 24, respectively. The analysis of HbA_{1c} responders using the CMH method showed a significant treatment difference versus placebo for the lixisenatide-treated group (p-value=0.0003 and p-value<0.0001, respectively) for both categories. At Week 24, 14.5% of lixisenatide-treated patients and 3.8% of placebo-treated patients had achieved HbA_{1c} values ≤6.5%: 28.3% of patients in the lixisenatide group and 12.0% of patients in the placebo group had achieved HbA1c values <7%.

TABLE 13
Number (%) of patients with HbA_{1c} value ≤6.5%
or <7% respectively at Week 24 - mITT population

| HbA1c | Placebo | Lixisenatide |
|---|---|---|
| (%) | (N = 165) | (N = 326) |
| Number | 158 | 304 |
| ≤6.5% | 6 (3.8%) | 44 (14.5%) |
| >6.5% | 152 (96.2%) | 260 (85.5%) |
| p-value vs. placebo^{(a)} | — | 0.0003 |
| Number | 158 | 304 |
| <7.0% | 19 (12.0%) | 86 (28.3%) |
| ≥7.0% | 139 (88.0%) | 218 (71.7%) |
| p-value vs. placebo^{(a)} | — | <0.0001 |

^{(a)}Cochran-Mantel-Haenszel (CMH) method stratified by randomization strata of screening HbA1c (<8.0 or ≥8.0%) and randomization strata of metformin use at screening (Yes or No).
The analysis included measurements obtained before the introduction of rescue medication and up to 3 days after the last dose of the double-blind investigational product injection on or before Visit 12 (Week 24), or Day 169 if Visit 12 (Week 24) is not available.

### 6.2.2 Secondary Efficacy Endpoints

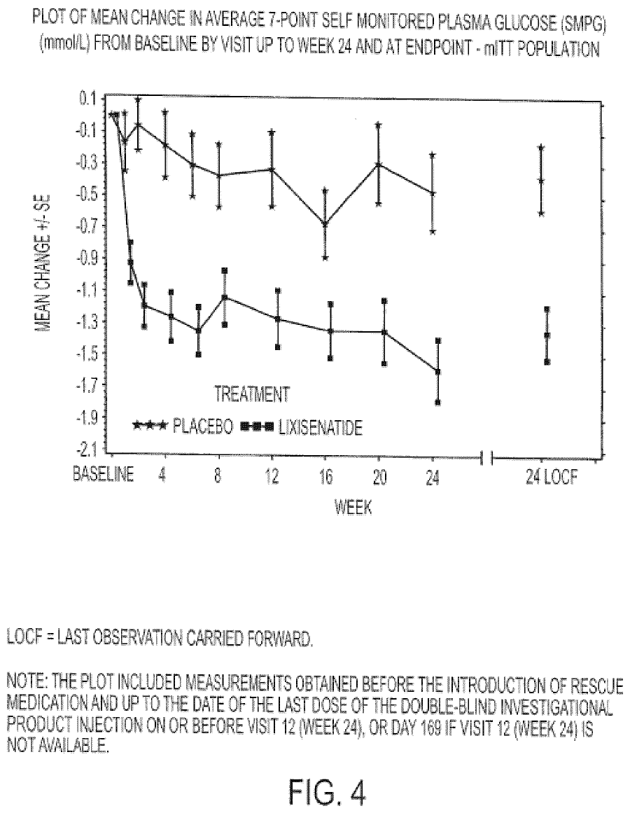

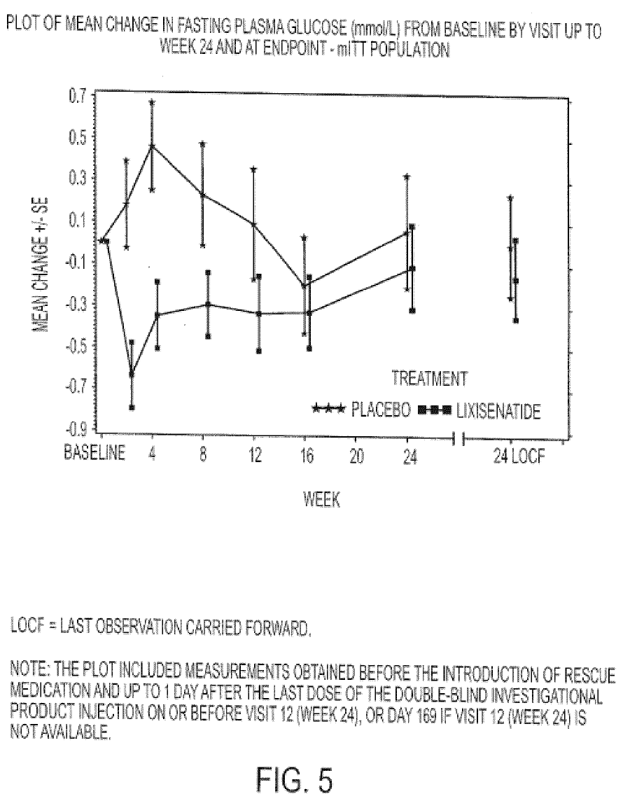

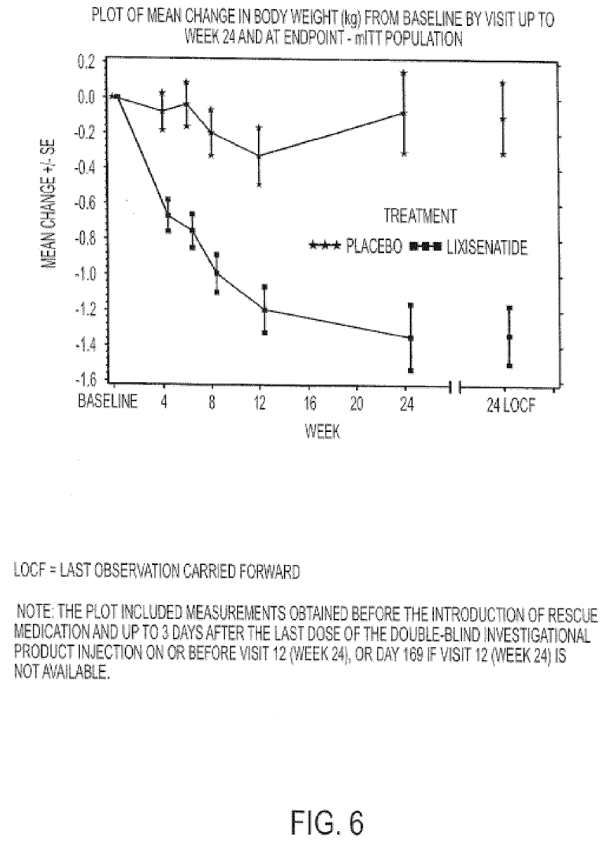

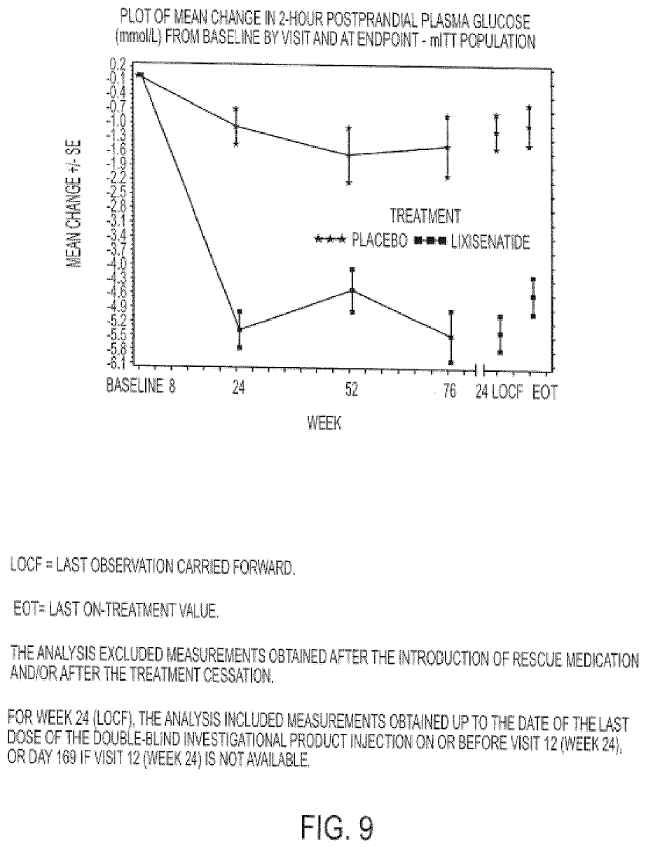

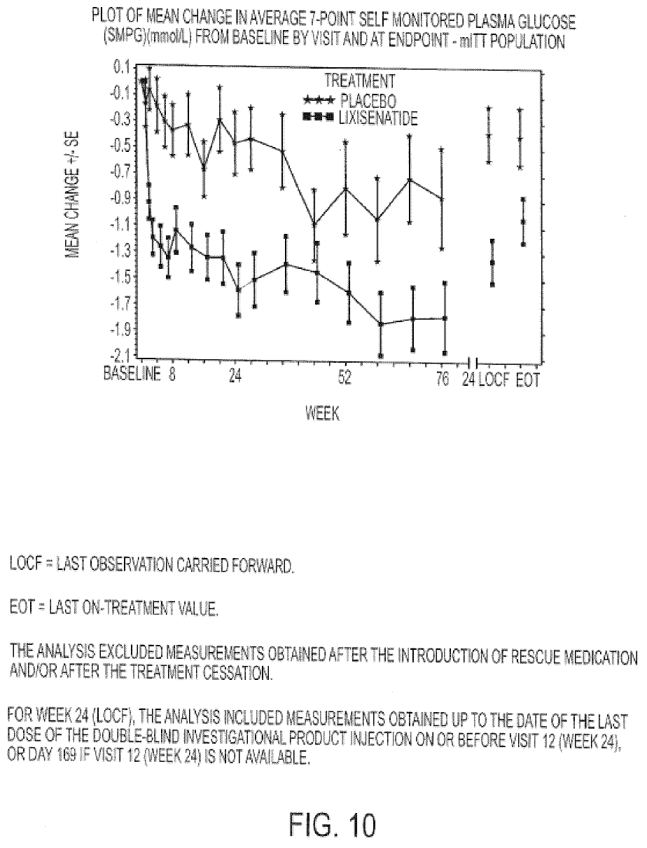

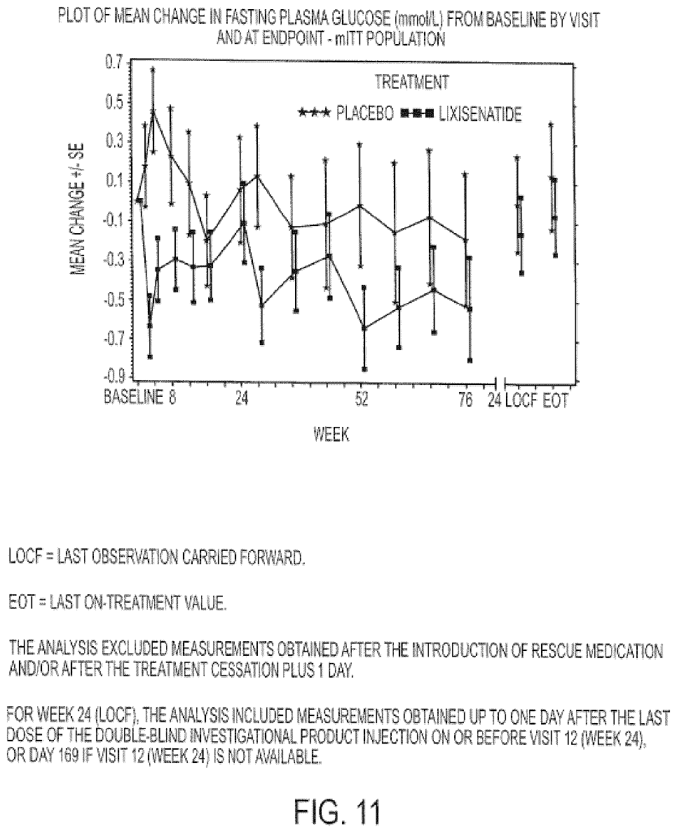

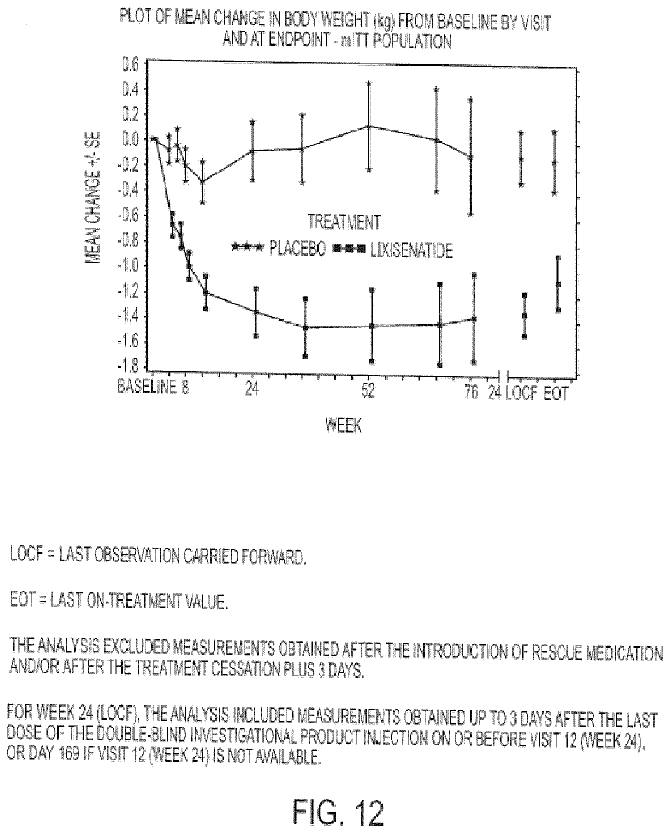

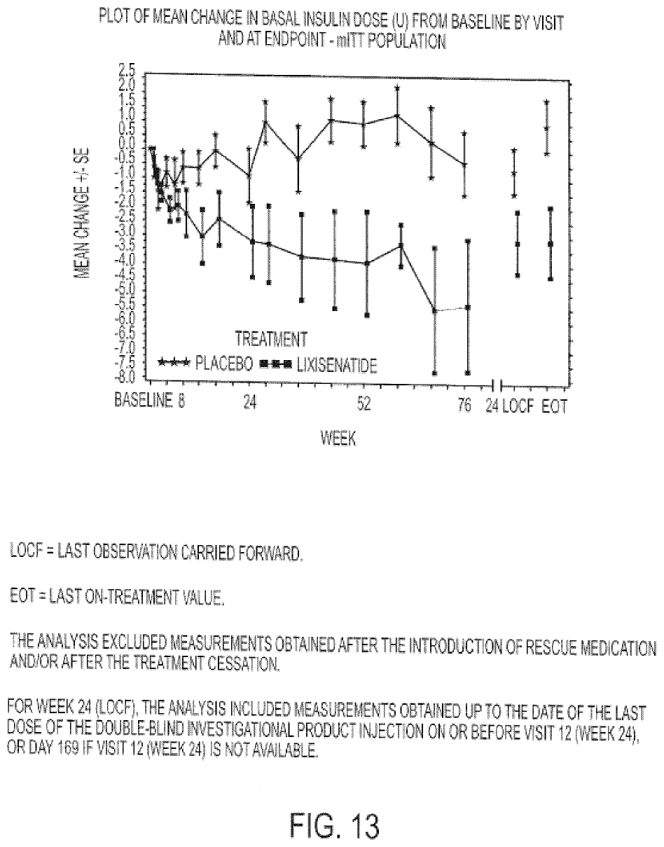

Table 14-18, and Table 20-21 summarize the ANCOVA analyses of 2-hour post-prandial plasma glucose, average 7-point SMPG, FPG, body weight, basal insulin, and glucose excursion respectively. FIG. 4-7 illustrate the Mean (±SE) change from baseline in average 7-point SMPG, FPG, body weight, and basal insulin over time during the main 24 week double-blind treatment period. FIG. 9-13 in the appendix illustrate the Mean (±SE) change from baseline in 2-hour post-prandial plasma glucose, average 7-point SMPG, FPG, body weight, and basal insulin over time up to Week 76.

The results of the 2-hour post-prandial plasma glucose after a test meal showed a statistically significant improvement from baseline to Week 24 in lixisenatide group compared with the placebo group (LS mean difference versus placebo=−3.81 mmol/L; p-value<0.0001). More over, treatment with lixisenatide substantially decreased post-prandial plasma glucose excursion from Baseline to Week 24 compared with the placebo group (LS mean difference=−3.80 mmol/L, 95% CI=−4.57 to −3.03) (Table 21).

For the average 7-point SMPG, a statistically significant improvement from baseline to Week 24 was observed in lixisenatide group compared with the placebo group (LS mean difference versus placebo=−0.88 mmol/L: p-value<0.0001) (Table 15).

Patients in both treatment groups showed a modest decrease in FPG from baseline to Week 24 (LSmean−0:63 for lixisenatide versus −0.55 for placebo) with no statistically significant difference observed between the lixisenatide and placebo group (LS mean difference versus placebo=−0.08 mmol/L; p-value=0.7579) (Table 16).

As per the testing strategy adjusting for multiplicity, inferential testing for body weight loss from baseline at Week 24 and the percentages of patients requiring rescue therapy at Week 24 were exploratory since the preceding test (FPG) failed to show statistically significant group difference.

The LS mean body weight loss from baseline to Week 24 was −1.80 kg for the lixisenatide-treated patients and −0.52 kg for the placebo-treated patients, with statistically significant difference observed between the two treatment groups (LS mean difference versus placebo 1.28 kg, p-value<0.0001) without an adjustment for multiplicity (Table 17). More lixisenatide-treated patients (132%) than placebo-treated patients (3.1%) had a weight loss of 5% or more from baseline to Week 24 (Table 18).

The percentages of patients requiring rescue therapy at Week 24 was slightly lower in the lixisenatide group than in the placebo group (18 patients [5.5%] in the lixisenatide group and 12 patients [7.3%] in the placebo group) (Table 19).

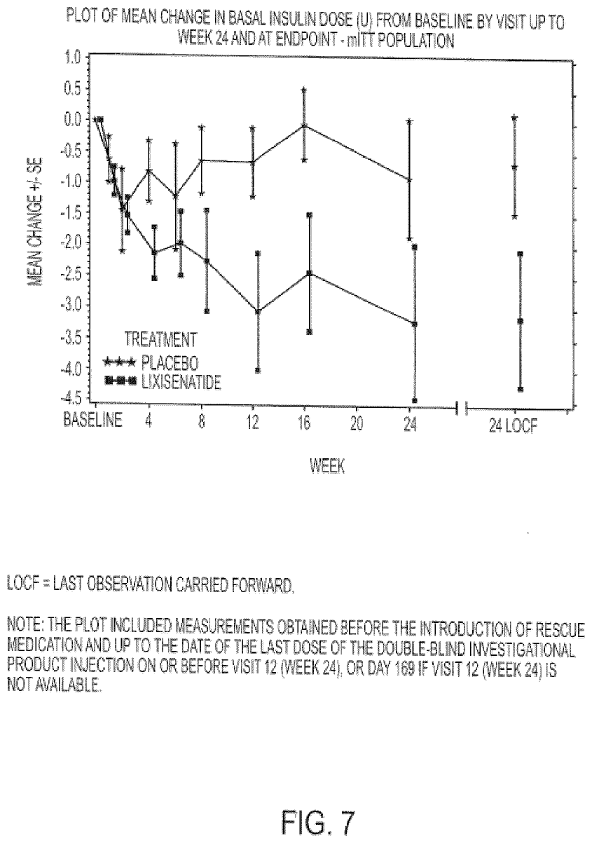

While achieving a greater HbA1C reduction, patients in lixisenatide group showed a steady reduction in daily basal insulin dose over the treatment period (FIG. 7) and reached a statistically significant decrease in mean change at the endpoint (Week 24) compared to the placebo group (LS mean difference versus placebo=−3.09 U; p-value=0.0412) (Table 20). The results from the analysis of the change in “total insulin dose” (not shown) are identical to those from the analysis on “basal insulin dose” due to the fact that the rescue insulin usage was excluded from the analysis.

TABLE 14
Mean change in 2-hour postprandial plasma glucose
(mmol/L) from baseline to Week 24 - mITT population

| 2-hour post-prandial | | |
|---|---|---|
| plasma glucose | Placebo | Lixisenatide |
| (mmol/L) | (N = 165) | (N = 326) |
| Baseline | | |
| Number | 123 | 235 |
| Mean (SD) | 15.85 (3.71) | 16.44 (4.29) |
| Median | 15.60 | 16.30 |
| Min:Max | 4.5:25.2 | 5.6:29.3 |
| Week 24 (LOCF) | | |
| Number | 123 | 235 |
| Mean (SD) | 14.73 (3.61) | 11.04 (4.38) |
| Median | 14.40 | 10.80 |
| Min:Max | 5.8:28.7 | 3.0:24.4 |
| Change from baseline to Week 24 | | |
| (LOCF) | | |
| Number | 123 | 235 |
| Mean (SD) | −1.13 (4.10) | −5.40 (5.76) |
| Median | −1.40 | −5.30 |
| Min:Max | −12.3:8.9 | −23.3:13.1 |
| LS Mean (SE) ^{(a)} | −1.72 (0.543) | −5.54 (0.468) |
| LS Mean difference (SE) vs. | — | −3.81 (0.451) |
| placebo ^{(a)} | | |
| 95% CI | — | (−4.699 to −2.925) |
| p-value | — | <.0001 |

LOCF = Last observation carried forward.
^{(a)} Analysis of covariance (ANCOVA) model with treatment groups (lixisenatide and placebo), randomization strata of screening HbA1c (<8.0, ≥8.0%), randomization strata of metformin use at screening (Yes, No), country as fixed effects and baseline 2-hour postprandial plasma glucose value as a covariate.
The analysis included measurements obtained before the introduction of rescue medication and up to the date of the last dose of the double-blind investigational product injection on or before Visit 12 (Week 24), or Day 169 if Visit 12 (Week 24) is not available. Patients with both baseline and Week 24 (LOCF) measurements are included.

TABLE 15
Mean change in average 7-point Self Monitored Plasma Glucose
(SMPG) (mmol/L) from baseline to Week 24 - mITT population

| Average 7-point SMPG | Placebo | Lixisenatide |
|---|---|---|
| (mmol/L) | (N = 165) | (N = 326) |
| Baseline | | |
| Number | 153 | 294 |
| Mean (SD) | 10.57 (2.69) | 10.74 (2.57) |
| Median | 10.16 | 10.53 |
| Min:Max | 6.8:23.4 | 5.3:21.4 |
| Week 24 (LOCF) | | |
| Number | 153 | 294 |
| Mean (SD) | 10.19 (2.44) | 9.39 (2.39) |
| Median | 9.74 | 9.17 |
| Min:Max | 5.9:21.7 | 5.2:20.6 |
| Change from baseline to Week 24 | | |
| (LOCF) | | |
| Number | 153 | 294 |
| Mean (SD) | −0.37 (2.55) | −1.35 (2.86) |
| Median | −0.36 | −1.12 |
| Min:Max | −11.2:5.0 | −9.7:8.4 |
| LS Mean (SE) ^{(a)} | −0.61 (0.238) | −1.49 (0.201) |
| LS Mean difference (SE) vs. | — | −0.88 (0.219) |
| placebo ^{(a)} | | |
| 95% CI | — | (−1.312 to −0.449) |
| p-value | — | <.0001 |

LOCF = Last observation carried forward.
^{(a)} Analysis of covariance (ANCOVA) model with treatment groups (lixisenatide and placebo), randomization strata of screening HbA1c (<8.0, ≥8.0%), randomization strata of metformin use at screening (Yes, No), country as fixed effects and baseline average 7-point SMPG value as a covariate.
The analysis included measurements obtained before the introduction of rescue medication and up to the date of the last dose of the double-blind investigational product injection on or before Visit 12 (Week 24), or Day 169 if Visit 12 (Week 24) is not available. Patients with both baseline and Week 24 (LOCF) measurements are included.

TABLE 16
Mean change in fasting plasma glucose (mmol/L)
from baseline to Week 24 - mITT population

| Fasting plasma glucose | Placebo | Lixisenatide |
|---|---|---|
| (mmol/L) | (N = 165) | (N = 326) |
| Baseline | | |
| Number | 163 | 317 |
| Mean (SD) | 8.03 (2.65) | 8.11 (2.84) |
| Median | 7.60 | 7.90 |
| Min:Max | 2.6:15.5 | 2.5:23.9 |
| Week 24 (LOCF) | | |
| Number | 163 | 317 |
| Mean (SD) | 8.02 (2.66) | 7.96 (2.97) |
| Median | 7.60 | 7.40 |
| Min:Max | 2.7:15.5 | 3.1:26.6 |
| Change from baseline to Week 24 | | |
| (LOCF) | | |
| Number | 163 | 317 |
| Mean (SD) | −0.00 (3.07) | −0.16 (3.41) |
| Median | 0.00 | −0.10 |
| Min:Max | −8.6:8.8 | −12.3:18.3 |
| LS Mean (SE) ^{(a)} | −0.55 (0.281) | −0.63 (0.233) |
| LS Mean difference (SE) vs. | — | −0.08 (0.259) |
| placebo ^{(a)} | | |
| 95% CI | — | (−0.590 to 0.430) |
| p-value | — | 0.7579 |

LOCF = Last observation carried forward.
^{(a)} Analysis of covariance (ANCOVA) model with treatment groups (lixisenatide and placebo), randomization strata of screening HbA1c (<8.0, ≥8.0%), randomization strata of metformin use at screening (Yes, No), country as fixed effects and baseline fasting plasma glucose as a covariate.
The analysis included measurements obtained before the introduction of rescue medication and up to 1 day after the last dose of the double-blind investigational product injection on or before Visit 12 (Week 24), or Day 169 if Visit 12 (Week 24) is not available. Patients with both baseline and Week 24 (LOCF) measurements are included.

TABLE 17
Mean change in body weight (kg) from
baseline to Week 24 - mITT population

| Body weight | Placebo | Lixisenatide |
|---|---|---|
| (kg) | (N = 165) | (N = 326) |
| Baseline | | |
| Number | 161 | 311 |
| Mean (SD) | 89.11 (21.00) | 87.39 (20.00) |
| Median | 85.90 | 85.70 |
| Min:Max | 50.2:160.5 | 46.5:157.8 |
| Week 24 (LOCF) | | |
| Number | 161 | 311 |
| Mean (SD) | 89.01 (20.86) | 86.05 (19.84) |
| Median | 84.50 | 84.00 |
| Min:Max | 52.0:160.0 | 46.0:158.5 |
| Change from baseline to Week 24 | | |
| (LOCF) | | |
| Number | 161 | 311 |
| Mean (SD) | −0.10 (2.57) | −1.33 (2.90) |
| Median | 0.00 | −1.00 |
| Min:Max | −10.9:9.4 | −11.5:6.7 |
| LS Mean (SE) ^{(a)} | −0.52 (0.293) | −1.80 (0.246) |
| LS Mean difference (SE) vs. | — | −1.28 (0.269) |
| placebo ^{(a)} | | |
| 95% CI | — | (−1.803 to −0.747) |
| p-value | — | <.0001 |

LOCF = Last observation carried forward.
^{(a)} Analysis of covariance (ANCOVA) model with treatment groups (lixisenatide and placebo), randomization strata of screening HbA1c (<8.0, ≥8.0%), randomization strata of metformin use at screening (Yes, No), country as fixed effects and baseline body weight as a covariate.
The analysis included measurements obtained before the introduction of rescue medication and up to 3 days after the last dose of the double-blind investigational product injection on or before Visit 12 (Week 24), or Day 169 if Visit 12 (Week 24) is not available. Patients with both baseline and Week 24 (LOCF) measurements are included.

TABLE 18
Number (%) of patients with >=5% weight loss
from baseline to Week 24 - mITT population

| | Placebo | Lixisenatide |
|---|---|---|
| Weight loss | (N = 165) | (N = 326) |
| Number | 161 | 311 |
| ≥5% | 5 (3.1%) | 41 (13.2%) |
| <5%^{(a)} | 156 (96.9%) | 270 (86.8%) |

^{(a)}Patients with less than 5% weight loss are included in this category, including patients who gained weight.
The analysis included measurements obtained before the introduction of rescue medication and up to 3 days after the last dose of the double-blind investigational product injection on or before Visit 12 (Week 24), or Day 169 if Visit 12 (Week 24) is not available. Patients with both baseline and Week 24 (LOCF) measurements are included.

TABLE 19
Number (%) of patients requiring rescue therapy during
the 24-week treatment period - mITT population

| | Placebo | Lixisenatide |
|---|---|---|
| Requiring rescue therapy | (N = 165) | (N = 326) |
| Number | 165 | 326 |
| Yes | 12 (7.3%) | 18 (5.5%) |
| No | 153 (92.7%) | 308 (94.5%) |
| p-value vs. placebo^{(a)} | — | 0.4433 |

^{(a)}Cochran-Mantel-Haenszel (CMH) method stratified by randomization strata of screening HbA1c (<8.0 or ≥8.0%) and metformin use at screening (Yes, No).

TABLE 20
Mean change in basal insulin dose (U) from
baseline to Week 24 - mITT population

| Daily basal insulin dose | Placebo | Lixisenatide |
|---|---|---|
| (U) | (N = 165) | (N = 326) |
| Baseline | | |
| Number | 165 | 325 |
| Mean (SD) | 57.65 (34.73) | 53.62 (33.97) |
| Median | 45.00 | 42.00 |
| Min:Max | 30.0:200.0 | 20.0:400.0 |
| Week 24 (LOCF) | | |
| Number | 165 | 325 |
| Mean (SD) | 56.99 (34.98) | 50.47 (28.09) |
| Median | 45.00 | 40.00 |
| Min:Max | 0.0:200.0 | 0.0:200.0 |
| Change from baseline to Week 24 | | |
| (LOCF) | | |
| Number | 165 | 325 |
| Mean (SD) | −0.66 (10.24) | −3.15 (19.62) |
| Median | 0.00 | 0.00 |
| Min:Max | −72.0:35.0 | −300.0:33.0 |
| LS Mean (SE) ^{(a)} | −1.93 (1.589) | −5.62 (1.317) |
| LS Mean difference (SE) vs. | — | −3.69 (1.464) |
| placebo ^{(a)} | | |
| 95% CI | — | (−6.568 to −0.815) |
| p-value | — | 0.0120 |

LOCF = Last observation carried forward.
^{(a)} Analysis of covariance (ANCOVA) model with treatment groups (lixisenatide and placebo), randomization strata of screening HbA1c (<8.0, ≥8.0%), randomization strata of metformin use at screening (Yes, No), country as fixed effects and baseline basal insulin dose as a covariate.
The analysis included measurements obtained before the introduction of rescue medication and up to the date of the last dose of the double-blind investigational product injection on or before Visit 12 (Week 24), or Day 169 if Visit 12 (Week 24) is not available. Patients with both baseline and Week 24 (LOCF) measurements are included.

TABLE 21
Mean change in glucose excursion (mmol/L)
from baseline to Week 24 - mITT population

| | Placebo | | Lixisenatide | |
|---|---|---|---|---|
| Glucose excursion(mmol/L) | (N = 165) | | (N = 326) | |
| Baseline | | | | |
| Number | 123 | | 233 | |
| Mean (SD) | 7.21 | (3.44) | 7.69 | (3.47) |
| Median | 7.20 | | 7.70 | |
| Min:Max | −4.7:15.0 | | −1.3:15.9 | |
| Week 24 (LOCF) | | | | |
| Number | 123 | | 233 | |
| Mean (SD) | 6.97 | (3.56) | 3.35 | (3.90) |
| Median | 6.70 | | 3.10 | |
| Min:Max | −0.2:20.2 | | −6.3:13.9 | |
| Change from baseline to | | | | |
| Week 24 (LOCF) | | | | |
| Number | 123 | | 233 | |
| Mean (SD) | −0.25 | (3.66) | −4.34 | (4.39) |
| Median | −0.40 | | −4.10 | |
| Min:Max | −11.6:11.1 | | −17.1:8.0 | |
| LS Mean (SE) ^{(a)} | −0.34 | (0.469) | −4.14 | (0.408) |

| | | | |
|---|---|---|---|
| LS Mean difference (SE) vs. | — | −3.80 | (0.392) |
| placebo ^{(a)} | | | |

| | | |
|---|---|---|
| 95% CI | — | (−4.572 to −3.031) |
| p-value | — | <.0001 |

LOCF = Last observation carried forward.
^{(a)} Analysis of covariance (ANCOVA) model with treatment groups (lixisenatide and placebo), randomization strata of screening HbA1c (<8.0, ≥8.0%), randomization strata of metformin use at screening (Yes, No), country as fixed effects and baseline glucose excursion value as a covariate.
Glucose excursion = 2-hour postprandial plasma glucose − plasma glucose 30 minutes prior to the meal test before study drug administration.
The analysis included measurements obtained before the introduction of rescue medication and up to the date of the last dose of the double-blind investigational product injection on or before Visit 12 (Week 24), or Day 169 if Visit 12 (Week 24) is not available. Patients with both baseline and Week 24 (LOCF) measurements are included.

### 6.3 Safety

An overview of the adverse events observed during the on-treatment period for the whole study is provided in Table 22. The proportions of the patients with treatment emergent adverse events (TEAEs) were generally comparable between the two treatment groups (87.5% for lixisenatide versus 85.6% for placebo). Four patients (2 inlixisenatide group and 2 in placebo) had TEAEs leading to death. The percentage of patients who experienced serious TEAEs was higher in the lixisenatide group (14.0%) than in the placebo group (10.2%). The percentage of patients with TEAEs leading to treatment discontinuation was 10.7% in the lixisenatide group compared with 7.2% in the placebo group. Tables 23, 24, and 25 summarize TEAEs leading to death, serious TEAEs, and TEAEs leading to treatment discontinuation by primary SOC, HLGT, HLT and PT, respectively. The most common TEAE leading to treatment discontinuation was nausea in the lixisenatide group (11 patients [3.4%]), while no patient in the placebo group discontinued the treatment due to nausea.

Table 35 in the appendix presents the incidences of TEAEs occurring in at least 1% of patients in any treatment group during the on-treatment period for the whole study. Hypoglycemia was the most frequently reported TEAE for both the lixisenatide (138 [42.1%]) and placebo groups (68 [40.7%]). Aside from hypoglycemia, the most common TEAE in the lixisenatide group was nausea (96 patients [29.3%] for lixisenatide versus 16 patients [9.6%] for placebo) followed by headache (41 patients [12.5%] for lixisenatide versus 17 [10.2%] for placebo) and diarrhea (37 patients [11.3%] for lixisenatide versus 10 [6.0%] for placebo).

TABLE 22
Overview of adverse event profile: treatment emergent
adverse events during the on-treatment period
for the whole study - Safety population

| | Placebo | Lixisenatide |
|---|---|---|
| | (N = 167) | (N = 328) |
| Patients with any TEAE | 143 (85.6%) | 287 (87.5%) |
| Patients with any serious TEAE | 17 (10.2%) | 46 (14.0%) |
| Patients with any TEAE leading to death | 2 (1.2%) | 2 (0.6%) |
| Patients with any TEAE leading to | 12 (7.2%) | 35 (10.7%) |
| permanent treatment discontinuation | | |

TEAE: Treatment emergent adverse event.
On-treatment period for the whole study = the time from the first dose of double-blind study medication up to 3 days after the last dose administration.
n (%) = number and percentage of patients with at least one adverse event.

TABLE 23
Number (%) of patients experiencing TEAE(s) leading
to death during the overall treatment period by primary
SOC, HLGT, HLT, and PT - Safety population

| PRIMARY SYSTEM ORGAN CLASS | | |
|---|---|---|
| HLGT: High Level Group Term | | |
| HLT: High Level Term | Placebo | Lixisenatide |
| Preferred Term | (N = 167) | (N = 328) |
| Any class | 2 (1.2%) | 2 (0.6%) |
| NEOPLASMS BENIGN, MALIGNANT AND | 1 (0.6%) | 0 |
| UNSPECIFIED (INCL CYSTS AND | | |
| POLYPS) | | |
| HLGT: Nervous system neoplasms malignant | 1 (0.6%) | 0 |
| and unspecified NEC | | |
| HLT: Nervous system neoplasms unspecified | 1 (0.6%) | 0 |
| malignancy NEC | | |
| Glioma | 1 (0.6%) | 0 |
| CARDIAC DISORDERS | 0 | 1 (0.3%) |
| HLGT: Coronary artery disorders | 0 | 1 (0.3%) |
| HLT: Ischaemic coronary artery disorders | 0 | 1 (0.3%) |
| Myocardial infarction | 0 | 1 (0.3%) |
| RESPIRATORY, THORACIC AND | 1 (0.6%) | 0 |
| MEDIASTINAL DISORDERS | | |
| HLGT: Respiratory disorders NEC | 1 (0.6%) | 0 |
| HLT: Respiratory failures (excl neonatal) | 1 (0.6%) | 0 |
| Respiratory failure | 1 (0.6%) | 0 |
| GENERAL DISORDERS AND | 0 | 1 (0.3%) |
| ADMINISTRATION SITE CONDITIONS | | |
| HLGT: Fatal outcomes | 0 | 1 (0.3%) |
| HLT: Death and sudden death | 0 | 1 (0.3%) |
| Sudden cardiac death | 0 | 1 (0.3%) |

TEAE: Treatment emergent adverse event, SOC: System Organ Class, HLGT: High Level Group Term, HLT: High Level Term, PT: Preferred Term.
On-treatment period for the whole study = the time from the first dose of double-blind study medication up to 3 days after the last dose administration.
MedDRA version: 13.1.
n (%) = number and percentage of patients with at least one TEAE leading to death.
Note:
Table sorted by SOC internationally agreed order and HLGT, HLT, PT alphabetic order.

TABLE 24
Number (%) of patients experiencing serious TEAE(s) during the overall treatment
period presented by primary SOC, HLGT, HLT, and PT - Safety population

| PRIMARY SYSTEM ORGAN CLASS | | |
|---|---|---|
| HLGT: High Level Group Term | | |
| HLT: High Level Term | Placebo | Lixisenatide |
| Preferred Term | (N = 167) | (N = 328) |
| Any class | 17 (10.2%) | 46 (14.0%) |
| INFECTIONS AND INFESTATIONS | 2 (1.2%) | 11 (3.4%) |
| HLGT: Bacterial infectious disorders | 1 (0.6%) | 1 (0.3%) |
| HLT: Bacterial infections NEC | 1 (0.6%) | 1 (0.3%) |
| Cellulitis | 0 | 1 (0.3%) |
| Incision site cellulitis | 1 (0.6%) | 0 |
| HLGT: Infections - pathogen unspecified | 2 (1.2%) | 8 (2.4%) |
| HLT: Infections NEC | 0 | 1 (0.3%) |
| Localised infection | 0 | 1 (0.3%) |
| HLT: Lower respiratory tract and lung infections | 2 (1.2%) | 3 (0.9%) |
| Bronchitis | 1 (0.6%) | 0 |
| Pneumonia | 2 (1.2%) | 3 (0.9%) |
| HLT: Sepsis, bacteraemia, viraemia and fungaemia NEC | 1 (0.6%) | 0 |
| Septic shock | 1 (0.6%) | 0 |
| HLT: Upper respiratory tract infections | 0 | 1 (0.3%) |
| Pharyngeal abscess | 0 | 1 (0.3%) |
| HLT: Urinary tract infections | 0 | 3 (0.9%) |
| Pyelonephritis | 0 | 1 (0.3%) |
| Urinary tract infection | 0 | 2 (0.6%) |
| HLGT: Viral infectious disorders | 0 | 3 (0.9%) |
| HLT: Coxsackie viral infections | 0 | 1 (0.3%) |
| Coxsackie viral infection | 0 | 1 (0.3%) |
| HLT: Papilloma viral infections | 0 | 1 (0.3%) |
| Anogenital warts | 0 | 1 (0.3%) |
| HLT: Viral infections NEC | 0 | 1 (0.3%) |
| Pneumonia viral | 0 | 1 (0.3%) |
| NEOPLASMS BENIGN, MALIGNANT AND UNSPECIFIED | 3 (1.8%) | 6 (1.8%) |
| (INCL CYSTS AND POLYPS) | | |
| HLGT: Breast neoplasms malignant and unspecified (incl nipple) | 0 | 1 (0.3%) |
| HLT: Breast and nipple neoplasms malignant | 0 | 1 (0.3%) |
| Breast cancer | 0 | 1 (0.3%) |
| HLGT: Gastrointestinal neoplasms malignant and unspecified | 0 | 1 (0.3%) |
| HLT: Pancreatic neoplasms malignant (excl islet cell and carcinoid) | 0 | 1 (0.3%) |
| Pancreatic carcinoma | 0 | 1 (0.3%) |
| HLGT: Nervous system neoplasms malignant and unspecified NEC | 1 (0.6%) | 0 |
| HLT: Nervous system neoplasms unspecified malignancy NEC | 1 (0.6%) | 0 |
| Glioma | 1 (0.6%) | 0 |
| HLGT: Renal and urinary tract neoplasms malignant and | 1 (0.6%) | 0 |
| unspecified | | |
| HLT: Renal neoplasms malignant | 1 (0.6%) | 0 |
| Renal cell carcinoma | 1 (0.6%) | 0 |
| HLGT: Reproductive neoplasms female malignant and unspecified | 0 | 1 (0.3%) |
| HLT: Cervix neoplasms malignant | 0 | 1 (0.3%) |
| Cervix carcinoma | 0 | 1 (0.3%) |
| HLGT: Reproductive neoplasms male malignant and unspecified | 1 (0.6%) | 0 |
| HLT: Prostatic neoplasms malignant | 1 (0.6%) | 0 |
| Prostate cancer | 1 (0.6%) | 0 |
| HLGT: Respiratory and mediastinal neoplasms malignant | 0 | 3 (0.9%) |
| and unspecified | | |
| HLT: Respiratory tract and pleural neoplasms malignant cell | 0 | 2 (0.6%) |
| type unspecified NEC | | |
| Lung cancer metastatic | 0 | 1 (0.3%) |
| Lung neoplasm malignant | 0 | 1 (0.3%) |
| HLT: Respiratory tract small cell carcinomas | 0 | 1 (0.3%) |
| Small cell lung cancer stage unspecified | 0 | 1 (0.3%) |
| BLOOD AND LYMPHATIC SYSTEM DISORDERS | 1 (0.6%) | 0 |
| HLGT: Platelet disorders | 1 (0.6%) | 0 |
| HLT: Thrombocytopenias | 1 (0.6%) | 0 |
| Thrombocytopenia | 1 (0.6%) | 0 |
| ENDOCRINE DISORDERS | 0 | 1 (0.3%) |
| HLGT: Thyroid gland disorders | 0 | 1 (0.3%) |
| HLT: Thyroid disorders NEC | 0 | 1 (0.3%) |
| Goitre | 0 | 1 (0.3%) |
| METABOLISM AND NUTRITION DISORDERS | 1 (0.6%) | 2 (0.6%) |
| HLGT: Glucose metabolism disorders (incl diabetes mellitus) | 1 (0.6%) | 2 (0.6%) |
| HLT: Hypoglycaemic conditions NEC | 1 (0.6%) | 2 (0.6%) |
| Hypoglycaemia | 1 (0.6%) | 1 (0.3%) |
| Hypoglycaemic unconsciousness | 0 | 1 (0.3%) |
| NERVOUS SYSTEM DISORDERS | 1 (0.6%) | 3 (0.9%) |
| HLGT: Central nervous system vascular disorders | 0 | 2 (0.6%) |
| HLT: Central nervous system vascular disorders NEC | 0 | 1 (0.3%) |
| Carotid artery stenosis | 0 | 1 (0.3%) |
| HLT: Transient cerebrovascular events | 0 | 1 (0.3%) |
| Transient ischaemic attack | 0 | 1 (0.3%) |
| HLGT: Headaches | 0 | 1 (0.3%) |
| HLT: Migraine headaches | 0 | 1 (0.3%) |
| Migraine | 0 | 1 (0.3%) |
| HLGT: Neurological disorders NEC | 1 (0.6%) | 2 (0.6%) |
| HLT: Disturbances in consciousness NEC | 0 | 1 (0.3%) |
| Somnolence | 0 | 1 (0.3%) |
| HLT: Neurological signs and symptoms NEC | 0 | 1 (0.3%) |
| Presyncope | 0 | 1 (0.3%) |
| HLT: Sensory abnormalities NEC | 1 (0.6%) | 0 |
| Intercostal neuralgia | 1 (0.6%) | 0 |
| EYE DISORDERS | 0 | 4 (1.2%) |
| HLGT: Anterior eye structural change, deposit and degeneration | 0 | 2 (0.6%) |
| HLT: Cataract conditions | 0 | 1 (0.3%) |
| Cataract | 0 | 1 (0.3%) |
| HLT: Lens structural change, deposit and degeneration | 0 | 1 (0.3%) |
| (excl cataracts) | | |
| Lens dislocation | 0 | 1 (0.3%) |
| HLGT: Retina, choroid and vitreous haemorrhages and | 0 | 2 (0.6%) |
| vascular disorders | | |
| HLT: Retinal bleeding and vascular disorders (excl retinopathy) | 0 | 1 (0.3%) |
| Retinal haemorrhage | 0 | 1 (0.3%) |
| HLT: Retinopathies NEC | 0 | 1 (0.3%) |
| Retinopathy haemorrhagic | 0 | 1 (0.3%) |
| CARDIAC DISORDERS | 9 (5.4%) | 5 (1.5%) |
| HLGT: Cardiac arrhythmias | 1 (0.6%) | 1 (0.3%) |
| HLT: Supraventricular arrhythmias | 1 (0.6%) | 1 (0.3%) |
| Atrial fibrillation | 1 (0.6%) | 0 |
| Atrial flutter | 0 | 1 (0.3%) |
| HLGT: Coronary artery disorders | 8 (4.8%) | 4 (1.2%) |
| HLT: Coronary artery disorders NEC | 4 (2.4%) | 2 (0.6%) |
| Coronary artery disease | 4 (2.4%) | 2 (0.6%) |
| HLT: Ischaemic coronary artery disorders | 6 (3.6%) | 2 (0.6%) |
| Acute myocardial infarction | 2 (1.2%) | 1 (0.3%) |
| Angina pectoris | 2 (1.2%) | 0 |
| Myocardial infarction | 1 (0.6%) | 1 (0.3%) |
| Myocardial ischaemia | 1 (0.6%) | 0 |
| HLGT: Myocardial disorders | 1 (0.6%) | 1 (0.3%) |
| HLT: Cardiomyopathies | 1 (0.6%) | 0 |
| Ischaemic cardiomyopathy | 1 (0.6%) | 0 |
| HLT: Myocardial disorders NEC | 0 | 1 (0.3%) |
| Left ventricular dysfunction | 0 | 1 (0.3%) |
| HLGT: Pericardial disorders | 1 (0.6%) | 0 |
| HLT: Pericardial disorders NEC | 1 (0.6%) | 0 |
| Pericardial effusion | 1 (0.6%) | 0 |
| VASCULAR DISORDERS | 0 | 2 (0.6%) |
| HLGT: Arteriosclerosis, stenosis, vascular insufficiency | 0 | 1 (0.3%) |
| and necrosis | | |
| HLT: Aortic necrosis and vascular insufficiency | 0 | 1 (0.3%) |
| Leriche syndrome | 0 | 1 (0.3%) |
| HLGT: Embolism and thrombosis | 0 | 1 (0.3%) |
| HLT: Peripheral embolism and thrombosis | 0 | 1 (0.3%) |
| Deep vein thrombosis | 0 | 1 (0.3%) |
| RESPIRATORY, THORACIC AND MEDIASTINAL DISORDERS | 2 (1.2%) | 0 |
| HLGT: Pulmonary vascular disorders | 1 (0.6%) | 0 |
| HLT: Pulmonary thrombotic and embolic conditions | 1 (0.6%) | 0 |
| Pulmonary embolism | 1 (0.6%) | 0 |
| HLGT: Respiratory disorders NEC | 1 (0.6%) | 0 |
| HLT: Respiratory failures (excl neonatal) | 1 (0.6%) | 0 |
| Respiratory failure | 1 (0.6%) | 0 |
| GASTROINTESTINAL DISORDERS | 0 | 3 (0.9%) |
| HLGT: Exocrine pancreas conditions | 0 | 1 (0.3%) |
| HLT: Acute and chronic pancreatitis | 0 | 1 (0.3%) |
| Pancreatitis | 0 | 1 (0.3%) |
| HLGT: Gastrointestinal haemorrhages NEC | 0 | 1 (0.3%) |
| HLT: Non-site specific gastrointestinal haemorrhages | 0 | 1 (0.3%) |
| Upper gastrointestinal haemorrhage | 0 | 1 (0.3%) |
| HLGT: Gastrointestinal ulceration and perforation | 0 | 1 (0.3%) |
| HLT: Gastric ulcers and perforation | 0 | 1 (0.3%) |
| Gastric ulcer | 0 | 1 (0.3%) |
| SKIN AND SUBCUTANEOUS TISSUE | 2 (1.2%) | 1 (0.3%) |
| DISORDERS | | |
| HLGT: Epidermal and dermal conditions | 1 (0.6%) | 0 |
| HLT: Dermatitis ascribed to specific agent | 1 (0.6%) | 0 |
| Toxic skin eruption | 1 (0.6%) | 0 |
| HLGT: Skin and subcutaneous tissue disorders NEC | 1 (0.6%) | 1 (0.3%) |
| HLT: Skin and subcutaneous tissue ulcerations | 1 (0.6%) | 1 (0.3%) |
| Neuropathic ulcer | 1 (0.6%) | 0 |
| Skin ulcer | 0 | 1 (0.3%) |
| MUSCULOSKELETAL AND CONNECTIVE | 0 | 2 (0.6%) |
| TISSUE DISORDERS | | |
| HLGT: Joint disorders | 0 | 1 (0.3%) |
| HLT: Osteoarthropathies | 0 | 1 (0.3%) |
| Osteoarthritis | 0 | 1 (0.3%) |
| HLGT: Musculoskeletal and connective tissue disorders NEC | 0 | 1 (0.3%) |
| HLT: Musculoskeletal and connective tissue pain and discomfort | 0 | 1 (0.3%) |
| Musculoskeletal chest pain | 0 | 1 (0.3%) |
| RENAL AND URINARY DISORDERS | 2 (1.2%) | 2 (0.6%) |
| HLGT: Bladder and bladder neck disorders (excl calculi) | 0 | 1 (0.3%) |
| HLT: Bladder neoplasms | 0 | 1 (0.3%) |
| Urinary bladder polyp | 0 | 1 (0.3%) |
| HLGT: Nephropathies | 1 (0.6%) | 0 |
| HLT: Nephropathies and tubular disorders NEC | 1 (0.6%) | 0 |
| Diabetic nephropathy | 1 (0.6%) | 0 |
| HLGT: Renal disorders (excl nephropathies) | 1 (0.6%) | 0 |
| HLT: Renal failure and impairment | 1 (0.6%) | 0 |
| Renal failure | 1 (0.6%) | 0 |
| HLGT: Urethral disorders (excl calculi) | 0 | 1 (0.3%) |
| HLT: Structural and obstructive urethral disorders (excl congenital) | 0 | 1 (0.3%) |
| Urethral stenosis | 0 | 1 (0.3%) |
| REPRODUCTIVE SYSTEM AND BREAST DISORDERS | 0 | 1 (0.3%) |
| HLGT: Prostatic disorders (excl infections and inflammations) | 0 | 1 (0.3%) |
| HLT: Prostatic neoplasms and hypertrophy | 0 | 1 (0.3%) |
| Benign prostatic hyperplasia | 0 | 1 (0.3%) |
| GENERAL DISORDERS AND ADMINISTRATION | 1 (0.6%) | 5 (1.5%) |
| SITE CONDITIONS | | |
| HLGT: Fatal outcomes | 0 | 1 (0.3%) |
| HLT: Death and sudden death | 0 | 1 (0.3%) |
| Sudden cardiac death | 0 | 1 (0.3%) |
| HLGT: General system disorders NEC | 1 (0.6%) | 4 (1.2%) |
| HLT: Pain and discomfort NEC | 1 (0.6%) | 4 (1.2%) |
| Non-cardiac chest pain | 1 (0.6%) | 4 (1.2%) |
| INJURY, POISONING AND PROCEDURAL COMPLICATIONS | 1 (0.6%) | 2 (0.6%) |
| HLGT: Bone and joint injuries | 1 (0.6%) | 1 (0.3%) |
| HLT: Spinal fractures and dislocations | 0 | 1 (0.3%) |
| Spinal compression fracture | 0 | 1 (0.3%) |
| HLT: Thoracic cage fractures and dislocations | 0 | 1 (0.3%) |
| Rib fracture | 0 | 1 (0.3%) |
| HLT: Upper limb fractures and dislocations | 1 (0.6%) | 0 |
| Wrist fracture | 1 (0.6%) | 0 |
| HLGT: Procedural related injuries and complications NEC | 0 | 1 (0.3%) |
| HLT: Non-site specific procedural complications | 0 | 1 (0.3%) |
| Procedural pain | 0 | 1 (0.3%) |
| SURGICAL AND MEDICAL PROCEDURES | 5 (3.0%) | 1 (0.3%) |
| HLGT: Vascular therapeutic procedures | 5 (3.0%) | 1 (0.3%) |
| HLT: Arterial therapeutic procedures (excl aortic) | 5 (3.0%) | 1 (0.3%) |
| Coronary angioplasty | 2 (1.2%) | 0 |
| Coronary arterial stent insertion | 1 (0.6%) | 0 |
| Coronary artery bypass | 2 (1.2%) | 1 (0.3%) |
| Coronary revascularisation | 1 (0.6%) | 0 |
| SOCIAL CIRCUMSTANCES | 1 (0.6%) | 0 |
| HLGT: Legal issues | 1 (0.6%) | 0 |
| HLT: Crime victims | 1 (0.6%) | 0 |
| Victim of crime | 1 (0.6%) | 0 |

TEAE: Treatment emergent adverse event, SOC: System Organ Class, HLGT: High Level Group Term, HLT: High Level Term, PT: Preferred Term.
On-treatment period for the whole study = the time from the first dose of double-blind study medication up to 3 days after the last dose administration.
MedDRA version: 13.1.
n (%) = number and percentage of patients with at least one serious TEAE.
Note:
Table sorted by SOC internationally agreed order and HLGT, HLT, PT alphabetic order.

TABLE 25
Number (%) of patients experiencing TEAE(s) leading to permanent
treatment discontinuation during the overall treatment period
by primary SOC, HLGT, HLT, and PT-Safety population

| PRIMARY SYSTEM ORGAN CLASS | | |
|---|---|---|
| HLGT: High Level Group Term | | |
| HLT: High Level Term | Placebo | Lixisenatide |
| Preferred Term | (N = 167) | (N = 328) |
| Any class | 12 (7.2%) | 35 (10.7%) |
| INFECTIONS AND INFESTATIONS | 1 (0.6%) | 1 (0.3%) |
| HLGT: Infections-pathogen unspecified | 1 (0.6%) | 1 (0.3%) |
| HLT: Abdominal and gastrointestinal infections | 0 | 1 (0.3%) |
| Gastroenteritis | 0 | 1 (0.3%) |
| HLT: Lower respiratory tract and lung infections | 1 (0.6%) | 0 |
| Pneumonia | 1 (0.6%) | 0 |
| HLT: Sepsis, bacteraemia, viraemia and fungaemia NEC | 1 (0.6%) | 0 |
| Septic shock | 1 (0.6%) | 0 |
| NEOPLASMS BENIGN, MALIGNANT AND UNSPECIFIED | 1 (0.6%) | 3 (0.9%) |
| (INCL CYSTS AND POLYPS) | | |
| HLGT: Breast neoplasms malignant and unspecified (incl nipple) | 0 | 1 (0.3%) |
| HLT: Breast and nipple neoplasms malignant | 0 | 1 (0.3%) |
| Breast cancer | 0 | 1 (0.3%) |
| HLGT: Gastrointestinal neoplasms malignant and unspecified | 0 | 1 (0.3%) |
| HLT: Pancreatic neoplasms malignant (excl islet cell and carcinoid) | 0 | 1 (0.3%) |
| Pancreatic carcinoma | 0 | 1 (0.3%) |
| HLGT: Nervous system neoplasms malignant and unspecified NEC | 1 (0.6%) | 0 |
| HLT: Nervous system neoplasms unspecified malignancy NEC | 1 (0.6%) | 0 |
| Glioma | 1 (0.6%) | 0 |
| HLGT: Respiratory and mediastinal neoplasms malignant | 0 | 1 (0.3%) |
| and unspecified | | |
| HLT: Respiratory tract small cell carcinomas | 0 | 1 (0.3%) |
| Small cell lung cancer stage unspecified | 0 | 1 (0.3%) |
| BLOOD AND LYMPHATIC SYSTEM DISORDERS | 1 (0.6%) | 0 |
| HLGT: Platelet disorders | 1 (0.6%) | 0 |
| HLT: Thrombocytopenias | 1 (0.6%) | 0 |
| Thrombocytopenia | 1 (0.6%) | 0 |
| IMMUNE SYSTEM DISORDERS | 0 | 2 (0.6%) |
| HLGT: Allergic conditions | 0 | 2 (0.6%) |
| HLT: Allergic conditions NEC | 0 | 2 (0.6%) |
| Hypersensitivity | 0 | 2 (0.6%) |
| METABOLISM AND NUTRITION DISORDERS | 0 | 3 (0.9%) |
| HLGT: Appetite and general nutritional disorders | 0 | 1 (0.3%) |
| HLT: Appetite disorders | 0 | 1 (0.3%) |
| Decreased appetite | 0 | 1 (0.3%) |
| HLGT: Glucose metabolism disorders (incl diabetes mellitus) | 0 | 2 (0.6%) |
| HLT: Hypoglycaemic conditions NEC | 0 | 2 (0.6%) |
| Hypoglycaemia | 0 | 1 (0.3%) |
| Hypoglycaemic unconsciousness | 0 | 1 (0.3%) |
| NERVOUS SYSTEM DISORDERS | 0 | 3 (0.9%) |
| HLGT: Central nervous system vascular disorders | 0 | 1 (0.3%) |
| HLT: Central nervous system vascular disorders NEC | 0 | 1 (0.3%) |
| Carotid arteriosclerosis | 0 | 1 (0.3%) |
| HLGT: Headaches | 0 | 1 (0.3%) |
| HLT: Headaches NEC | 0 | 1 (0.3%) |
| Headache | 0 | 1 (0.3%) |
| HLGT: Neurological disorders NEC | 0 | 1 (0.3%) |
| HLT: Neurological signs and symptoms NEC | 0 | 1 (0.3%) |
| Dizziness | 0 | 1 (0.3%) |
| EYE DISORDERS | 0 | 1 (0.3%) |
| HLGT: Retina, choroid and vitreous haemorrhages and | 0 | 1 (0.3%) |
| vascular disorders | | |
| HLT: Retinopathies NEC | 0 | 1 (0.3%) |
| Retinopathy haemorrhagic | 0 | 1 (0.3%) |
| EAR AND LABYRINTH DISORDERS | 0 | 1 (0.3%) |
| HLGT: Inner ear and VIIIth cranial nerve disorders | 0 | 1 (0.3%) |
| HLT: Inner ear signs and symptoms | 0 | 1 (0.3%) |
| Tinnitus | 0 | 1 (0.3%) |
| Vertigo | 0 | 1 (0.3%) |
| CARDIAC DISORDERS | 4 (2.4%) | 2 (0.6%) |
| HLGT: Cardiac arrhythmias | 1 (0.6%) | 0 |
| HLT: Supraventricular arrhythmias | 1 (0.6%) | 0 |
| Atrial fibrillation | 1 (0.6%) | 0 |
| HLGT: Coronary artery disorders | 4 (2.4%) | 2 (0.6%) |
| HLT: Coronary artery disorders NEC | 2 (1.2%) | 1 (0.3%) |
| Coronary artery disease | 2 (1.2%) | 1 (0.3%) |
| HLT: Ischaemic coronary artery disorders | 2 (1.2%) | 1 (0.3%) |
| Acute myocardial infarction | 2 (1.2%) | 0 |
| Myocardial infarction | 0 | 1 (0.3%) |
| HLGT: Myocardial disorders | 0 | 1 (0.3%) |
| HLT: Myocardial disorders NEC | 0 | 1 (0.3%) |
| Left ventricular dysfunction | 0 | 1 (0.3%) |
| VASCULAR DISORDERS | 0 | 1 (0.3%) |
| HLGT: Arteriosclerosis, stenosis, vascular insufficiency | 0 | 1 (0.3%) |
| and necrosis | | |
| HLT: Aortic necrosis and vascular insufficiency | 0 | 1 (0.3%) |
| Leriche syndrome | 0 | 1 (0.3%) |
| RESPIRATORY, THORACIC AND MEDIASTINAL DISORDERS | 1 (0.6%) | 0 |
| HLGT: Respiratory disorders NEC | 1 (0.6%) | 0 |
| HLT: Respiratory failures (excl neonatal) | 1 (0.6%) | 0 |
| Respiratory failure | 1 (0.6%) | 0 |
| GASTROINTESTINAL DISORDERS | 2 (1.2%) | 17 (5.2%) |
| HLGT: Exocrine pancreas conditions | 0 | 1 (0.3%) |
| HLT: Acute and chronic pancreatitis | 0 | 1 (0.3%) |
| Pancreatitis | 0 | 1 (0.3%) |
| HLGT: Gastrointestinal haemorrhages NEC | 0 | 1 (0.3%) |
| HLT: Non-site specific gastrointestinal haemorrhages | 0 | 1 (0.3%) |
| Upper gastrointestinal haemorrhage | 0 | 1 (0.3%) |
| HLGT: Gastrointestinal motility and defaecation conditions | 0 | 4 (1.2%) |
| HLT: Diarrhoea (excl infective) | 0 | 3 (0.9%) |
| Diarrhoea | 0 | 3 (0.9%) |
| HLT: Gastrointestinal atonic and hypomotility disorders NEC | 0 | 1 (0.3%) |
| Gastrooesophageal reflux disease | 0 | 1 (0.3%) |
| HLGT: Gastrointestinal signs and symptoms | 1 (0.6%) | 13 (4.0%) |
| HLT: Dyspeptic signs and symptoms | 0 | 2 (0.6%) |
| Dyspepsia | 0 | 2 (0.6%) |
| HLT: Flatulence, bloating and distension | 0 | 1 (0.3%) |
| Abdominal distension | 0 | 1 (0.3%) |
| HLT: Gastrointestinal and abdominal pains (excl oral and throat) | 0 | 1 (0.3%) |
| Abdominal pain | 0 | 1 (0.3%) |
| HLT: Gastrointestinal signs and symptoms NEC | 1 (0.6%) | 0 |
| Faecal incontinence | 1 (0.6%) | 0 |
| HLT: Nausea and vomiting symptoms | 0 | 11 (3.4%) |
| Nausea | 0 | 11 (3.4%) |
| Vomiting | 0 | 2 (0.6%) |
| HLGT: Tongue conditions | 1 (0.6%) | 0 |
| HLT: Tongue signs and symptoms | 1 (0.6%) | 0 |
| Tongue oedema | 1 (0.6%) | 0 |
| SKIN AND SUBCUTANEOUS TISSUE | 0 | 2 (0.6%) |
| DISORDERS | | |
| HLGT: Epidermal and dermal conditions | 0 | 1 (0.3%) |
| HLT: Erythemas | 0 | 1 (0.3%) |
| Rash erythematous | 0 | 1 (0.3%) |
| HLGT: Skin appendage conditions | 0 | 1 (0.3%) |
| HLT: Apocrine and eccrine gland disorders | 0 | 1 (0.3%) |
| Hyperhidrosis | 0 | 1 (0.3%) |
| MUSCULOSKELETAL AND CONNECTIVE | 1 (0.6%) | 0 |
| TISSUE DISORDERS | | |
| HLGT: Musculoskeletal and connective tissue deformities | 1 (0.6%) | 0 |
| (incl intervertebral disc disorders) | | |
| HLT: Spine and neck deformities | 1 (0.6%) | 0 |
| Spondylolisthesis | 1 (0.6%) | 0 |
| RENAL AND URINARY DISORDERS | 2 (1.2%) | 2 (0.6%) |
| HLGT: Nephropathies | 1 (0.6%) | 1 (0.3%) |
| HLT: Nephropathies and tubular disorders NEC | 1 (0.6%) | 1 (0.3%) |
| Diabetic nephropathy | 1 (0.6%) | 1 (0.3%) |
| HLGT: Renal disorders (excl nephropathies) | 1 (0.6%) | 1 (0.3%) |
| HLT: Renal failure and impairment | 1 (0.6%) | 1 (0.3%) |
| Renal failure | 1 (0.6%) | 0 |
| Renal failure acute | 0 | 1 (0.3%) |
| PREGNANCY, PUERPERIUM AND PERINATAL CONDITIONS | 1 (0.6%) | 0 |
| HLGT: Pregnancy, labour, delivery and postpartum conditions | 1 (0.6%) | 0 |
| HLT: Normal pregnancy, labour and delivery | 1 (0.6%) | 0 |
| Pregnancy | 1 (0.6%) | 0 |
| GENERAL DISORDERS AND ADMINISTRATION SITE | 0 | 3 (0.9%) |
| CONDITIONS | | |
| HLGT: Fatal outcomes | 0 | 1 (0.3%) |
| HLT: Death and sudden death | 0 | 1 (0.3%) |
| Sudden cardiac death | 0 | 1 (0.3%) |
| HLGT: General system disorders NEC | 0 | 2 (0.6%) |
| HLT: Asthenic conditions | 0 | 2 (0.6%) |
| Asthenia | 0 | 2 (0.6%) |
| INVESTIGATIONS | 2 (1.2%) | 1 (0.3%) |
| HLGT: Endocrine investigations (incl sex hormones) | 1 (0.6%) | 1 (0.3%) |
| HLT: Gastrointestinal, pancreatic and APUD hormone analyses | 1 (0.6%) | 1 (0.3%) |
| Blood calcitonin increased | 1 (0.6%) | 1 (0.3%) |
| HLGT: Hepatobiliary investigations | 1 (0.6%) | 0 |
| HLT: Liver function analyses | 1 (0.6%) | 0 |
| Liver function test abnormal | 1 (0.6%) | 0 |

TEAE: Treatment emergent adverse event,
SOC: System Organ Class,
HLGT: High Level Group Term,
HLT: High Level Term,
PT: Preferred Term.
On-treatment period for the whole study = the time from the first dose of double-blind study medication up to 3 days after the last dose administration.
MedDRA version: 13.1.
n (%) = number and percentage of patients with at least one TEAE leading to permanent treatment discontinuation.
Note:
Table sorted by SOC internationally agreed order and HLGT, HLT, PT alphabetic order.

According to the protocol definition for symptomatic hypoglycemia, 138 (42.1%) lixisenatide-treated patients and 65 (38.9%) placebo-treated patients reported at least one symptomatic hypoglycemic event during the on-treatment period for the whole study (Table 26). Of these patients having the symptomatic hypoglycemia events per protocol definition, 4 lixisenatide-treated patients had investigator reported AE terms other than hypoglycemia (hypoglycaemic unconsciousness, hypoglycemia unawareness, blood glucose decreased, and tremor), which are not displayed as the hypoglycemia PT in the TEAE summary table (Table 35). Conversely, 7 patients (4 for lixisenatide and 3 +or placebo) who reported hypoglycemia TEAEs are excluded from the protocol defined symptomatic hypoglycemia events in Table 26 because of either not fulfilling the hypoglycemia per protocol definition (the event glucose values 260 mg/dL) or missing relevant information for analysis (the complementary form was missing for one placebo-treated patient).

Seven (2.1%) lixisenatide-treated patients reported 8 severe symptomatic hypoglycemia events per protocol definition, whereas 1 (0.6%) placebo-treated patients reported 1 severe symptomatic hypoglycemia event during the same period (Table 27).

TABLE 26
Summary of symptomatic hypoglycemia during the on-treatment
period for the whole study-Safety population

| | Placebo | Lixisenatide |
|---|---|---|
| Type | (N = 167) | (N = 328) |
| Total patient years | 231.22 | 442.71 |
| Any symptomatic hypoglycemia | | |
| Number of patients with events, n (%) | 65 (38.9%) | 138 (42.1%) |
| Number of patients with events per | 28.1 | 31.2 |
| 100 patient years¹ | | |
| Blood glucose <60 mg/dL | | |
| Number of patients with events, n (%) | 64 (38.3%) | 134 (40.9%) |
| Number of patients with events per | 27.7 | 30.3 |
| 100 patient years¹ | | |
| No blood glucose reported | | |
| Number of patients with events, n (%) | 2 (1.2%) | 20 (6.1%) |
| Number of patients with events per | 0.9 | 4.5 |
| 100 patient years¹ | | |

Symptomatic hypoglycemia = symptomatic hypoglycemia as defined per protocol.
On-treatment period for the whole study = the time from the first dose of double-blind study medication up to 3 days after the last dose administration.
¹Calculated as (number of patients with events*100 divided by total exposure + 3 days in patient years).

TABLE 27
Summary of severe symptomatic hypoglycemia during the on-
treatment period for the whole study-Safety population

| | Placebo | Lixisenatide |
|---|---|---|
| Type | (N = 167) | (N = 328) |
| Total patient years | 231.22 | 442.71 |
| Any severe symptomatic hypoglycemia | | |
| Number of patients with events, n (%) | 1 (0.6%) | 7 (2.1%) |
| Number of patients with events per | 0.4 | 1.6 |
| 100 patient years¹ | | |
| Blood glucose <36 mg/dL | | |
| Number of patients with events, n (%) | 1 (0.6%) | 5 (1.5%) |
| Number of patients with events per | 0.4 | 1.1 |
| 100 patient years¹ | | |
| No blood glucose reported | | |
| Number of patients with events, n (%) | 0 | 2 (0.6%) |
| Number of patients with events per | 0 | 0.5 |
| 100 patient years¹ | | |

Severe symptomatic hypoglycemia = severe symptomatic hypoglycemia as defined per protocol.
On-treatment period for the whole study = the time from the first dose of double-blind study medication up to 3 days after the last dose administration.
¹Calculated as (number of patients with events*100 divided by total exposure + 3 days in patient years).

Eight patients (2.4%) from lixisenatide group and one patient (0.6%) from placebo group experienced injection site reaction AEs (Table 28). The injection site reaction AEs were identified by searching for the term “injection site” in either the investigator reported AE PTs or PTs from the ARAC diagnosis after the allergic reaction adjudication. None of the reactions were serious or severe in intensity, nor did these AEs lead to IP discontinuation.

TABLE 28
Number (%) of patients experiencing injection site reactions during
the on-treatment period for the whole study-Safety population

| Event Source | Placebo | Lixisenatide |
|---|---|---|
| Preferred Term | (N = 167) | (N = 328) |
| Any injection site reactions | 1 (0.6%) | 8 (2.4%) |
| Investigator reported PTs | 1 (0.6%) | 7 (2.1%) |
| Injection site haematoma | 1 (0.6%) | 0 |
| Injection site haemorrhage | 0 | 1 (0.3%) |
| Injection site induration | 0 | 1 (0.3%) |
| Injection site infection | 0 | 1 (0.3%) |
| Injection site nodule | 0 | 1 (0.3%) |
| Injection site pain | 0 | 2 (0.6%) |
| Injection site pruritus | 0 | 1 (0.3%) |
| PTs by ARAC diagnosis | 0 | 1 (0.3%) |
| Injection site reaction | 0 | 1 (0.3%) |

On-treatment period for the whole study = the time from the first dose of double-blind study medication up to 3 days after the last dose administration.
ARAC = Allergic Reaction Assessment Committee.

A total of 33 cases were reported for 28 patients as suspected allergic events by investigators and sent to ARAC for adjudication during the on-treatment period for the whole study. Of these, 11 events from 11 patients (8 [2.4%] lixisenatide-treated patients and 3 [1.8%] placebo-treated patients) were adjudicated as allergic reactions by the ARAC, but only 3 events from 3 patients (two anaphylactic reaction events from lixisenatide group and one angioedema event from placebo group) were adjudicated as possibly related to the IP (Table 29).

Patient 840635031 (lixisenatide): A 52-year-old female patient with a medical history of dyslipidemia, asthma, allergic rhinitis, allergies to drug, food, pollen and dust, as well as urticaria and angioedema in the past, reported a mild injection site reactions with each dose since 8 Jun. 2009(21 days on the IP). The patient complained of local and generalized itching, and swelling at the injection site with erythema. She also presented with hoarseness, change in pitch of voice, wheezing and chest tightness. The vital signs during a reaction (17 Jun. 2009, 9:08) were: BP129/62 mmHg, HR 67 bpm. She was treated with oral Benadryl and recovered on 18 Jun. 2009. The IP was discontinued on 17 Jun. 2009. The causal assessment was related per the investigator. The allergic reaction was adjudicated as anaphylactic reaction and possibly related to the IP by the ARAC.

Patient 840635033 (lixisenatide): A 58-year-old female patient with a medical history of hypertension, dyslipidemia, asthma, allergic rhinitis and allergies to drug and house dust, as well as rash in the past, developed pruritus and urticaria of severe intensity on 19 Jul. 2009 (25 days on the IP) following administration with the IP and oral metoclopramide, (newly started on 19 Jul. 2009 and stopped on 20 Jul. 2009). The patient complained of generalized itching, flushing, swelling in lips, eyes and face, nasal congestion and feeling nausea. The vital signs during a reaction (23 Jul. 2009, 10:10) were: BP134/66 mmHg, HR 95 bpm. She was treated with oral Benadryl and rapidly improved. The IP was discontinued on 23 Jul. 2009. The causal assessment was related to the IP but also possibly to metoclopramide per the investigator. The allergic reaction was adjudicated as anaphylactic reaction and possibly related to the IP by the ARAC.

TABLE 29
Number (%) of patients with events adjudicated as allergic reaction by ARAC
during the on-treatment period of the whole study-Safety population

| | MedDRA coded | | | |
|---|---|---|---|---|
| Relationship to | term (PT) | | | |
| study treatment | for ARAC | | Placebo | Lixisenatide |
| (by ARAC) | diagnosis | ARAC diagnosis | (N = 167) | (N = 328) |
| All | Events adjudicated as | | 3 (1.8%) | 8 (2.4%) |
| | allergic reaction by ARAC | | | |
| | Anaphylactic reaction | ANAPHYLACTIC | 0 | 2 (0.6%) |
| | | REACTION | | |
| | Angioedema | ANGIOEDEMA | 2 (1.2%) | 0 |
| | Asthma | ASTHMA | 0 | 1 (0.3%) |
| | | EXACERBATION | | |
| | Dermatitis contact | ALLERGIC CONTACT | 0 | 1 (0.3%) |
| | | DERMATITIS | | |
| | Dermatitis | DRUG-INDUCED | 1 (0.6%) | 0 |
| | | DERMATITIS | | |
| | Pruritus | PRURITUS | 0 | 1 (0.3%) |
| | Rhinitis allergic | ALLERGIC RHINITIS | 0 | 1 (0.3%) |
| | Urticaria | URTICARIA (HIVES) | 0 | 2 (0.6%) |
| Possibly | Events adjudicated as | | 1 (0.6%) | 2 (0.6%) |
| Related | allergic reaction by ARAC | | | |
| to IP | Anaphylactic reaction | ANAPHYLACTIC | 0 | 2 (0.6%) |
| | | REACTION | | |
| | Angioedema | ANGIOEDEMA | 1 (0.6%) | 0 |
| Not related | Events adjudicated as | | 2 (1.2%) | 6 (1.8%) |
| to IP | allergic reaction by ARAC | | | |
| | Angioedema | ANGIOEDEMA | 1 (0.6%) | 0 |
| | Asthma | ASTHMA | 0 | 1 (0.3%) |
| | | EXACERBATION | | |
| | Dermatitis contact | ALLERGIC CONTACT | 0 | 1 (0.3%) |
| | | DERMATITIS | | |
| | Dermatitis | DRUG-INDUCED | 1 (0.6%) | 0 |
| | | DERMATITIS | | |
| | Pruritus | PRURITUS | 0 | 1 (0.3%) |
| | Rhinitis allergic | ALLERGIC RHINITIS | 0 | 1 (0.3%) |
| | Urticaria | URTICARIA (HIVES) | 0 | 2 (0.6%) |

ARAC = Allergic Reaction Assessment Committee.
IP = Investigational product.
On-treatment period for the whole study = the time from the first dose of double-blind study medication up to 3 days after the last dose administration.

Per protocol, any increase in amylase and/or lipase above twice the upper limit of normal range (ULN) that had been confirmed by a repeat measurement was to be monitored and documented on a pre-specified form: “adverse event form for suspected pancreatitis”. During the on-treatment period for the whole study, 6 (1.8%) lixisenatide-treated patients and 1 (0.6%) placebo-treated patient reported 7 TEAEs with the pre-specified AE form (Table 30). Among these 7 patients, one lixisenatide-treated patient (#840614004) had an AE of pancreatitis.

Patient 840614004 (lixisenatide): A 60-year-old male patient with a medical history of prostatic hyperplasia, dislocated lumbar disc (L4), acid reflux, hypertension, depression, liver resection due to liver abscess and chronic smoking, was on insulin glargine, metformin and other concomitant medications including anti-hypertensive, antacids, and anti-inflammatory and pain medications.

On 5 Apr. 2010 (272 days on the IP), the patient was hospitalized for pancreatitis of severe intensity and the IP was discontinued. The patient complained of on-and-off recurrent diarrhea associated with nausea, vomiting and cramping epigastric pain for one week. The amylase was 166 IU/L and lipase was 40 U/L. His symptoms improved and he was discharged from the hospital on 8 Apr. 2010, but his lipase continued to increase. On 6 May 2010, the patient had a gastroenterology evaluation and a computed tomography (CT) revealed dilatation of the pancreatic ducts, apparently due to chronic pancreatitis. On 24 Jun. 2010, 11 weeks-after the last dose of the IP, the patient developed severe exacerbation of pancreatitis and was hospitalized. The patient presented with acute abdominal pain with multiple episodes of vomiting. The patient's amylase level was 159 U/L and lipase was 1108 U/L. The patient recovered from the pancreatitis exacerbation on 27 Jun. 2010 and the causal assessment was not related to the IP per the investigator.

Patients who had at least one value of lipase or amylase3 ULN during the on-treatment period are summarized in Table 31. Ten patients (7 [2.2%] patients in the lixisenatide group and 3 [1.8%] in the placebo group) with elevated lipase (≥3ULN) were observed. One patient in the lixisenatide group had elevated amylase (≥3ULN), and none did in the placebo group.

TABLE 30
Number (%) of patients with TEAE suspected pancreatitis during
the on-treatment period for the whole study-Safety population

| | Placebo | Lixisenatide |
|---|---|---|
| Preferred Term | (N = 167) | (N = 328) |
| Any | 1 (0.6%) | 6 (1.8%) |
| Hyperamylasaemia | 0 | 1 (0.3%) |
| Lipase increased | 1 (0.6%) | 3 (0.9%) |
| Pancreatic enzymes increased | 0 | 1 (0.3%) |
| Pancreatitis | 0 | 1 (0.3%) |

On-treatment period for the whole study = the time from the first dose of double-blind study medication up to 3 days after the last dose administration.
n (%) = number and percentage of patients with any cases reported on the AE form for suspected pancreatitis along with complementary form.

TABLE 31
Pancreatic enzymes: Number (%) of patients with at least one
post-baseline PCSA during the on-treatment period for the whole
study according to baseline status-Safety population

| Laboratory criteria | | |
|---|---|---|
| Baseline | Placebo | Lixisenatide |
| By PCSA criteria n/N1 (%) | (N = 167) | (N = 328) |
| Lipase | | |
| Total* | | |
| ≥3 ULN | 3/163 (1.8%) | 7/321 (2.2%) |
| Normal/Missing | | |
| ≥3 ULN | 3/162 (1.9%) | 7/321 (2.2%) |
| Total* | | |
| ≥3 ULN | 0/163 | 1/321 (0.3%) |
| Normal/Missing | | |
| ≥3 ULN | 0/163 | 1/321 (0.3%) |

Note:
PCSA: Potentially Clinically Significant Abnormalities,
ULN = Upper limit of normal.
On-treatment period for the whole study = the time from the first dose of double-blind study medication up to 3 days after the last dose administration.
*Regardless of baseline.
Note:
The number (n) represents the subset of the total number of patients who met the criterion in question at least once. The denominator (/N1) for each parameter within a treatment group is the number of patients for the treatment group who had that parameter assessed post-baseline by baseline PCSA status. Only the worsening of the worst case for each patient is presented by baseline status.

### Amylass

Per protocol, any calcitonin value >20 pg/mL confirmed by a repeat measurement was to be monitored and reported on the pre-specified adverse event form for “increased calcitonin ≥20pg/mL”. During the on-treatment period for whole study, 5 patients (4 [1.2%] for lixisenatide and 1 [0.6%] for placebo) reported 5 TEAEs of blood calcitonin increase (Table 32). Of these 5 patients, 1 lixisenatide-treated patient (#840636032) had calcitonin values 250 ng/L and 4 others had calcitonin values ≥20 ng/L but <50 ng/L. In addition, one placebo-treated patient had a TEAE of “blood calcitonin increased” reported by the investigator (Table 35) although the values were <20ng/L. This AE was not documented in the pre-specified AE form per protocol and therefore is not included in Table 32. A description of the case with calcitonin value ≥50 ng/L is provided below:

Patient 840636032 (lixisenatide): A 55-year-old male patient with a medical history of depression, dyslipidemia and hypertension was on insulin glargine, metformin and other concomitant medications including multivitamins, gemfibrozil, valsartan, bupropion, fluoxetine and methylphenidate. On 24 Sep. 2009 (Week 24), an AE of “blood calcitonin increased” was reported for a value of 47.5 ng/L, which was the result of the first calcitonin measurement for this patient. The AE was not accompanied with any symptoms. Calcitonin rose to 75.9 and 70.7 ng/L in January and February 2010 so the IP was discontinued. On 23 Feb. 2010, the patient saw a thyroid specialist and underwent a thyroid ultrasound scan, which revealed a “normal” result. The follow-up calcitonin (27 May 2010) was 50 ng/L. A repeat thyroid ultrasound (28 Jul. 2010) was again “normal”. The thyroid specialist assessed the event as “an incidentally discovered elevation in calcitonin level is of unknown significance” and recommended serial monitoring of calcitonin through time and a repeat ultrasound in one year. The causal relationship was not related to the IP per the investigator. A post-study calcitonin on 23 Dec. 2010 was 60 pg/mL (local laboratory reference <11 pg/mL).

TABLE 32
Number (%) of patients with TEAE increased calcitonin during the
on-treatment period for the whole study-Safety population

| | Placebo | Lixisenatide |
|---|---|---|
| Preferred Term | (N = 167) | (N = 328) |
| Any | 1 (0.6%) | 4 (1.2%) |
| Blood calcitonin increased | 1 (0.6%) | 4 (1.2%) |

On-treatment period for the whole study = the time from the first dose of double-blind study medication up to 3 days after the last dose administration.
n (%) = number and percentage of patients with any cases reported on the AE form for increased calcitonin ≥20 ng/mL.

Patients with at least one serum calcitonin measured during the on-treatment period of the whole study are summarized in Table 33 according to the 4 categories of calcitonin level at baseline. Eight (2.8%) patients in the lixisenatide group and 1 (0.7%) patient in the placebo group had calcitonin values ≥20 ng/L (Table 33) including those 5 patients (4 on lixisenatide) who reported a TEAE with the pre-specified AE form (Table 32). Four out of the 8 lixisenatide-treated patients with a calcitonin value ≥20 ng/L (Table 33) did not report a TEAE with the pre-specified AE form because of an unconfirmed elevation; 2 had a single value ≥50 ng/L (all other measurements <20ng/L) and 2 had a value between ≥20-<50 ng/L. It should be pointed out that calcitonin measurement was introduced into the protocol via a protocol amendment after most patients had been randomized. Therefore, baseline values are missing for the majority of the patients (209 [88.2%] for lixisenatide and 101 [88.6%] for placebo).

TABLE 33
Serum calcitonin-Number (%) of patients by pre-defined
categories during the on-treatment period of the whole
study according to baseline category-Safety population

| Laboratory criteria | | |
|---|---|---|
| Baseline status | Placebo | Lixisenatide |
| Post-baseline | (N = 167) | (N = 328) |
| Calcitonin (ng/L) | | |
| Total* | | |
| ≤ULN | 127/147 (86.4%) | 246/281 (87.5%) |
| >ULN-<20 ng/L | 19/147 (12.9%) | 27/281 (9.6%) |
| ≥20 ng/L-<50 ng/L | 1/147 (0.7%) | 5/281 (1.8%) |
| ≥50 ng/L | 0/147 | 3/281 (1.1%) |
| Missing | | |
| ≤ULN | 101/114 (88.6%) | 209/237 (88.2%) |
| >ULN-<20 ng/L | 13/114 (11.4%) | 22/237 (9.3%) |
| ≥20 ng/L-<50 ng/L | 0/114 | 3/237 (1.3%) |
| ≥50 ng/L | 0/114 | 3/237 (1.3%) |
| ≤ULN | | |
| ≤ULN | 26/29 (89.7%) | 37/39 (94.9%) |
| >ULN-<20 ng/L | 3/29 (10.3%) | 2/39 (5.1%) |
| ≥20 ng/L-<50 ng/L | 0/29 | 0/39 |
| ≥50 ng/L | 0/29 | 0/39 |
| >ULN-<20 ng/L | | |
| ≤ULN | 0/4 | 0/4 |
| >ULN-<20 ng/L | 3/4 (75.0%) | 3/4 (75.0%) |
| ≥20 ng/L-<50 ng/L | 1/4 (25.0%) | 1/4 (25.0%) |
| ≥50 ng/L | 0/4 | 0/4 |
| ≥20 ng/L-<50 ng/L | | |
| ≤ULN | 0/0 | 0/1 |
| >ULN-<20 ng/L | 0/0 | 0/1 |
| ≥20 ng/L-<50 ng/L | 0/0 | 1/1 (100%) |
| ≥50 ng/L | 0/0 | 0/1 |
| ≥50 ng/L | | |
| ≤ULN | 0/0 | 0/0 |
| >ULN-<20 ng/L | 0/0 | 0/0 |
| ≥20 ng/L-<50 ng/L | 0/0 | 0/0 |
| ≥50 ng/L | 0/0 | 0/0 |

ULN = Upper limit of normal.
On-treatment period for the whole study = the time from the first dose of double-blind study medication up to 3 days after the last dose administration.
*Regardless of baseline.
Note:
The numerator represents the number of patients who were in the pre-specified categories in each baseline category. The denominator for each parameter within a treatment group is the number of patients for the treatment group who had that parameter assessed post-baseline by baseline status.
A patient is counted only in the worst category.

TABLE 34
Mean change in HbA1c (%) from baseline by visit - mITT population

| Treatment | Observed data | | | | | | | Change from baseline | | | | | | |
|---|---|---|---|---|---|---|---|---|---|---|---|---|---|---|
| Time point | N | Mean | SD | SE | Median | Min | Max | N | Mean | SD | SE | Median | Min | Max |
| Placebo | | | | | | | | | | | | | | |
| (N = 165) | | | | | | | | | | | | | | |
| Screening | 165 | 8.46 | 0.81 | 0.063 | 8.50 | 7.0 | 10.0 | | | | | | | |
| Baseline | 165 | 8.37 | 0.84 | 0.065 | 8.40 | 6.7 | 10.5 | | | | | | | |
| Week 8 | 147 | 8.09 | 1.02 | 0.084 | 8.00 | 6.1 | 11.6 | 147 | −0.31 | 0.68 | 0.056 | −0.30 | −2.1 | 1.4 |
| Week 12 | 145 | 8.04 | 1.12 | 0.093 | 7.90 | 5.9 | 11.6 | 145 | −0.33 | 0.85 | 0.070 | −0.30 | −2.5 | 1.9 |
| Week 24 | 134 | 8.07 | 1.16 | 0.101 | 7.80 | 6.0 | 11.6 | 134 | −0.28 | 1.04 | 0.090 | −0.40 | −2.7 | 2.7 |
| Week 24 | 158 | 8.13 | 1.17 | 0.093 | 7.90 | 6.0 | 11.6 | 158 | −0.24 | 0.98 | 0.078 | −0.30 | −2.7 | 2.7 |
| (LOCF) | | | | | | | | | | | | | | |
| Week 36 | 115 | 7.98 | 1.08 | 0.101 | 7.90 | 5.6 | 11.3 | 115 | −0.32 | 1.01 | 0.094 | −0.30 | −3.0 | 3.0 |
| Week 44 | 103 | 7.83 | 1.00 | 0.099 | 7.70 | 6.0 | 11.7 | 103 | −0.40 | 0.95 | 0.094 | −0.40 | −2.9 | 2.9 |
| Week 52 | 94 | 7.71 | 0.99 | 0.102 | 7.65 | 5.7 | 12.1 | 94 | −0.47 | 0.98 | 0.101 | −0.45 | −2.8 | 3.6 |
| Week 60 | 81 | 7.70 | 1.15 | 0.128 | 7.50 | 5.8 | 12.3 | 81 | −0.50 | 1.08 | 0.119 | −0.50 | −3.1 | 4.5 |
| Week 68 | 76 | 7.64 | 1.02 | 0.117 | 7.50 | 5.8 | 11.2 | 76 | −0.50 | 1.03 | 0.118 | −0.50 | −2.9 | 2.5 |
| Week 76 | 67 | 7.69 | 1.00 | 0.123 | 7.40 | 6.0 | 11.6 | 67 | −0.43 | 1.04 | 0.127 | −0.40 | −2.8 | 2.9 |
| Week 84 | 29 | 7.82 | 1.19 | 0.221 | 7.80 | 5.7 | 11.2 | 29 | −0.35 | 0.99 | 0.184 | −0.40 | −1.7 | 2.4 |
| Week 92 | 21 | 7.87 | 1.06 | 0.232 | 7.70 | 5.7 | 10.2 | 21 | −0.20 | 1.07 | 0.235 | 0.10 | −1.8 | 2.1 |
| Week 100 | 11 | 7.98 | 0.87 | 0.261 | 7.60 | 7.1 | 9.6 | 11 | 0.02 | 0.84 | 0.254 | −0.10 | −1.3 | 1.4 |
| Week 108 | 5 | 7.76 | 0.30 | 0.133 | 7.80 | 7.3 | 8.1 | 5 | −0.22 | 0.40 | 0.180 | −0.20 | −0.8 | 0.2 |
| Last on- | 158 | 8.24 | 1.12 | 0.089 | 8.20 | 5.7 | 12.0 | 158 | −0.13 | 0.96 | 0.076 | −0.10 | −2.8 | 2.7 |
| treatment | | | | | | | | | | | | | | |
| value | | | | | | | | | | | | | | |
| Lixisenatide | | | | | | | | | | | | | | |
| (N = 326) | | | | | | | | | | | | | | |
| Screening | 326 | 8.49 | 0.83 | 0.046 | 8.50 | 7.0 | 10.0 | | | | | | | |
| Baseline | 326 | 8.42 | 0.88 | 0.049 | 8.40 | 6.0 | 10.8 | | | | | | | |
| Week 8 | 286 | 7.68 | 1.00 | 0.059 | 7.60 | 5.5 | 14.1 | 286 | −0.72 | 0.86 | 0.051 | −0.70 | −2.8 | 5.7 |
| Week 12 | 272 | 7.54 | 0.94 | 0.057 | 7.40 | 5.5 | 10.9 | 272 | −0.84 | 0.86 | 0.052 | −0.90 | −3.0 | 2.5 |
| Week 24 | 251 | 7.65 | 1.11 | 0.070 | 7.50 | 5.6 | 11.3 | 251 | −0.72 | 1.01 | 0.064 | −0.80 | −3.5 | 2.9 |
| Week 24 | 304 | 7.76 | 1.18 | 0.068 | 7.60 | 5.6 | 14.1 | 304 | −0.63 | 1.08 | 0.062 | −0.60 | −3.5 | 5.7 |
| (LOCF) | | | | | | | | | | | | | | |
| Week 36 | 220 | 7.60 | 1.09 | 0.073 | 7.40 | 5.5 | 12.0 | 220 | −0.74 | 1.01 | 0.068 | −0.80 | −3.6 | 2.6 |
| Week 44 | 191 | 7.52 | 1.11 | 0.080 | 7.50 | 5.3 | 11.7 | 191 | −0.75 | 1.07 | 0.078 | −0.80 | −3.8 | 2.9 |
| Week 52 | 183 | 7.49 | 1.16 | 0.086 | 7.40 | 5.3 | 11.8 | 183 | −0.74 | 1.10 | 0.081 | −0.80 | −3.5 | 2.9 |
| Week 60 | 163 | 7.50 | 1.18 | 0.092 | 7.30 | 5.4 | 12.4 | 163 | −0.71 | 1.16 | 0.091 | −0.70 | −3.5 | 4.0 |
| Week 68 | 155 | 7.43 | 1.19 | 0.095 | 7.30 | 5.1 | 12.5 | 155 | −0.76 | 1.14 | 0.092 | −0.80 | −3.1 | 4.0 |
| Week 76 | 150 | 7.39 | 1.15 | 0.094 | 7.30 | 5.3 | 13.7 | 150 | −0.79 | 1.16 | 0.094 | −0.95 | −2.9 | 5.5 |
| Week 84 | 87 | 7.30 | 1.04 | 0.111 | 7.30 | 5.3 | 10.9 | 87 | −0.90 | 1.10 | 0.118 | −1.00 | −3.2 | 2.4 |
| Week 92 | 58 | 7.06 | 0.92 | 0.120 | 7.05 | 5.0 | 9.5 | 58 | −1.05 | 0.94 | 0.124 | −1.10 | −2.9 | 1.4 |
| Week 100 | 29 | 6.91 | 0.93 | 0.173 | 6.80 | 5.5 | 9.6 | 29 | −0.98 | 0.96 | 0.179 | −1.00 | −3.0 | 0.9 |
| Week 108 | 18 | 6.77 | 0.85 | 0.201 | 6.65 | 5.4 | 8.6 | 18 | −1.01 | 0.97 | 0.228 | −1.10 | −2.5 | 1.4 |
| Week 116 | 6 | 7.05 | 1.26 | 0.516 | 7.05 | 5.5 | 9.0 | 6 | −0.68 | 1.01 | 0.414 | −0.75 | −2.3 | 0.7 |
| Week 124 | 2 | 7.15 | 1.77 | 1.250 | 7.15 | 5.9 | 8.4 | 2 | −0.45 | 0.78 | 0.550 | −0.45 | −1.0 | 0.1 |
| Last on- | 304 | 7.96 | 1.18 | 0.067 | 7.90 | 5.3 | 14.1 | 304 | −0.44 | 1.10 | 0.063 | −0.45 | −3.1 | 5.7 |
| treatment | | | | | | | | | | | | | | |
| value | | | | | | | | | | | | | | |

LOCF = Last observation carried forward.
The analysis excluded measurements obtained after the introduction of rescue medication and/or after the treatment cessation plus 3 days.
For Week 24 (LOCF), the analysis included measurements obtained up to 3 days after the last dose of the double-blind investigational product injection on or before Visit 12 (Week 24), or Day 169 if Visit 12 (Week 24) is not available.

TABLE 35
Number (%) of patients experiencing common TEAE(s) (PT ≥1%
in any treatment group) during the overall treatment period
presented by primary SOC, HLGT, HLT and PT-Safety population

| PRIMARY SYSTEM ORGAN CLASS | | |
|---|---|---|
| HLGT: High Level Group Term | | |
| HLT: High Level Term | Placebo | Lixisenatide |
| Preferred Term | (N = 167) | (N = 328) |
| Any class | 143 (85.6%) | 287 (87.5%) |
| INFECTIONS AND INFESTATIONS | 72 (43.1%) | 148 (45.1%) |
| HLGT: Fungal infectious disorders | 3 (1.8%) | 12 (3.7%) |
| HLT: Fungal infections NEC | 3 (1.8%) | 8 (2.4%) |
| Fungal skin infection | 2 (1.2%) | 0 |
| Onychomycosis | 0 | 4 (1.2%) |
| HLGT: Infections-pathogen unspecified | 60 (35.9%) | 125 (38.1%) |
| HLT: Abdominal and gastrointestinal infections | 5 (3.0%) | 14 (4.3%) |
| Gastroenteritis | 4 (2.4%) | 12 (3.7%) |
| HLT: Dental and oral soft tissue infections | 4 (2.4%) | 7 (2.1%) |
| Tooth infection | 2 (1.2%) | 5 (1.5%) |
| HLT: Ear infections | 3 (1.8%) | 6 (1.8%) |
| Ear infection | 2 (1.2%) | 0 |
| HLT: Infections NEC | 3 (1.8%) | 5 (1.5%) |
| Localised infection | 2 (1.2%) | 3 (0.9%) |
| HLT: Lower respiratory tract and lung infections | 15 (9.0%) | 26 (7.9%) |
| Bronchitis | 13 (7.8%) | 22 (6.7%) |
| Pneumonia | 2 (1.2%) | 5 (1.5%) |
| HLT: Upper respiratory tract infections | 40 (24.0%) | 84 (25.6%) |
| Laryngitis | 3 (1.8%) | 0 |
| Nasopharyngitis | 21 (12.6%) | 32 (9.8%) |
| Pharyngitis | 5 (3.0%) | 12 (3.7%) |
| Pharyngotonsillitis | 3 (1.8%) | 1 (0.3%) |
| Rhinitis | 2 (1.2%) | 3 (0.9%) |
| Sinusitis | 5 (3.0%) | 12 (3.7%) |
| Upper respiratory tract infection | 6 (3.6%) | 24 (7.3%) |
| HLT: Urinary tract infections | 7 (4.2%) | 26 (7.9%) |
| Urinary tract infection | 6 (3.6%) | 21 (6.4%) |
| HLGT: Viral infectious disorders | 22 (13.2%) | 41 (12.5%) |
| HLT: Herpes viral infections | 3 (1.8%) | 1 (0.3%) |
| Herpes zoster | 3 (1.8%) | 1 (0.3%) |
| HLT: Influenza viral infections | 9 (5.4%) | 28 (8.5%) |
| Influenza | 9 (5.4%) | 28 (8.5%) |
| HLT: Viral infections NEC | 11 (6.6%) | 11 (3.4%) |
| Gastroenteritis viral | 4 (2.4%) | 4 (1.2%) |
| Respiratory tract infection viral | 5 (3.0%) | 3 (0.9%) |
| NEOPLASMS BENIGN, MALIGNANT AND UNSPECIFIED | 7 (4.2%) | 11 (3.4%) |
| (INCL CYSTS AND POLYPS) | | |
| HLGT: Endocrine neoplasms malignant and unspecified | 2 (1.2%) | 2 (0.6%) |
| HLT: Endocrine neoplasms malignant and unspecified NEC | 2 (1.2%) | 2 (0.6%) |
| Thyroid neoplasm | 2 (1.2%) | 2 (0.6%) |
| BLOOD AND LYMPHATIC SYSTEM DISORDERS | 6 (3.6%) | 9 (2.7%) |
| HLGT: Anaemias nonhaemolytic and marrow depression | 5 (3.0%) | 7 (2.1%) |
| HLT: Anaemias NEC | 4 (2.4%) | 6 (1.8%) |
| Anaemia | 3 (1.8%) | 6 (1.8%) |
| METABOLISM AND NUTRITION DISORDERS | 72 (43.1%) | 151 (46.0%) |
| HLGT: Appetite and general nutritional disorders | 3 (1.8%) | 13 (4.0%) |
| HLT: Appetite disorders | 3 (1.8%) | 13 (4.0%) |
| Decreased appetite | 2 (1.2%) | 11 (3.4%) |
| HLGT: Glucose metabolism disorders (incl diabetes mellitus) | 68 (40.7%) | 141 (43.0%) |
| HLT: Hypoglycaemic conditions NEC | 68 (40.7%) | 141 (43.0%) |
| Hypoglycaemia | 68 (40.7%) | 138 (42.1%) |
| Hypoglycaemia unawareness | 2 (1.2%) | 9 (2.7%) |
| HLGT: Lipid metabolism disorders | 2 (1.2%) | 8 (2.4%) |
| HLT: Elevated triglycerides | 2 (1.2%) | 6 (1.8%) |
| Hypertriglyceridaemia | 2 (1.2%) | 6 (1.8%) |
| HLGT: Purine and pyrimidine metabolism disorders | 1 (0.6%) | 7 (2.1%) |
| HLT: Purine metabolism disorders NEC | 1 (0.6%) | 7 (2.1%) |
| Hyperuricaemia | 0 | 5 (1.5%) |
| PSYCHIATRIC DISORDERS | 13 (7.8%) | 25 (7.6%) |
| HLGT: Anxiety disorders and symptoms | 7 (4.2%) | 11 (3.4%) |
| HLT: Anxiety symptoms | 7 (4.2%) | 10 (3.0%) |
| Anxiety | 4 (2.4%) | 7 (2.1%) |
| Stress | 3 (1.8%) | 0 |
| HLGT: Depressed mood disorders and disturbances | 3 (1.8%) | 8 (2.4%) |
| HLT: Depressive disorders | 3 (1.8%) | 8 (2.4%) |
| Depression | 3 (1.8%) | 8 (2.4%) |
| HLGT: Sleep disorders and disturbances | 5 (3.0%) | 5 (1.5%) |
| HLT: Disturbances in initiating and maintaining sleep | 5 (3.0%) | 4 (1.2%) |
| Insomnia | 5 (3.0%) | 4 (1.2%) |
| NERVOUS SYSTEM DISORDERS | 48 (28.7%) | 95 (29.0%) |
| HLGT: Headaches | 17 (10.2%) | 44 (13.4%) |
| HLT: Headaches NEC | 17 (10.2%) | 41 (12.5%) |
| Headache | 17 (10.2%) | 41 (12.5%) |
| HLT: Migraine headaches | 0 | 4 (1.2%) |
| Migraine | 0 | 4 (1.2%) |
| HLGT: Movement disorders (incl parkinsonism) | 6 (3.6%) | 18 (5.5%) |
| HLT: Tremor (excl congenital) | 6 (3.6%) | 18 (5.5%) |
| Tremor | 6 (3.6%) | 18 (5.5%) |
| HLGT: Neurological disorders NEC | 19 (11.4%) | 45 (13.7%) |
| HLT: Disturbances in consciousness NEC | 0 | 6 (1.8%) |
| Somnolence | 0 | 5 (1.5%) |
| HLT: Neurological signs and symptoms NEC | 11 (6.6%) | 28 (8.5%) |
| Dizziness | 11 (6.6%) | 26 (7.9%) |
| HLT: Paraesthesias and dysaesthesias | 5 (3.0%) | 10 (3.0%) |
| Paraesthesia | 4 (2.4%) | 10 (3.0%) |
| HLT: Sensory abnormalities NEC | 6 (3.6%) | 4 (1.2%) |
| Hypoaesthesia | 2 (1.2%) | 3 (0.9%) |
| Restless legs syndrome | 2 (1.2%) | 0 |
| HLGT: Peripheral neuropathies | 9 (5.4%) | 10 (3.0%) |
| HLT: Chronic polyneuropathies | 6 (3.6%) | 3 (0.9%) |
| Diabetic neuropathy | 6 (3.6%) | 3 (0.9%) |
| HLT: Mononeuropathies | 1 (0.6%) | 5 (1.5%) |
| Carpal tunnel syndrome | 1 (0.6%) | 4 (1.2%) |
| HLGT: Spinal cord and nerve root disorders | 2 (1.2%) | 7 (2.1%) |
| HLT: Lumbar spinal cord and nerve root disorders | 2 (1.2%) | 7 (2.1%) |
| Sciatica | 2 (1.2%) | 7 (2.1%) |
| EYE DISORDERS | 18 (10.8%) | 38 (11.6%) |
| HLGT: Anterior eye structural change, deposit and degeneration | 4 (2.4%) | 9 (2.7%) |
| HLT: Cataract conditions | 4 (2.4%) | 8 (2.4%) |
| Cataract | 4 (2.4%) | 8 (2.4%) |
| HLGT: Eye disorders NEC | 4 (2.4%) | 1 (0.3%) |
| HLT: Ocular disorders NEC | 4 (2.4%) | 0 |
| Eye pain | 4 (2.4%) | 0 |
| HLGT: Glaucoma and ocular hypertension | 0 | 5 (1.5%) |
| HLT: Glaucomas (excl congenital) | 0 | 5 (1.5%) |
| Glaucoma | 0 | 5 (1.5%) |
| HLGT: Ocular infections, irritations and inflammations | 2 (1.2%) | 7 (2.1%) |
| HLT: Conjunctival infections, irritations and inflammations | 2 (1.2%) | 4 (1.2%) |
| Conjunctivitis | 2 (1.2%) | 4 (1.2%) |
| HLGT: Retina, choroid and vitreous haemorrhages and | 4 (2.4%) | 10 (3.0%) |
| vascular disorders | | |
| HLT: Retinopathies NEC | 4 (2.4%) | 8 (2.4%) |
| Diabetic retinopathy | 3 (1.8%) | 6 (1.8%) |
| HLGT: Vision disorders | 4 (2.4%) | 12 (3.7%) |
| HLT: Visual disorders NEC | 3 (1.8%) | 9 (2.7%) |
| Vision blurred | 2 (1.2%) | 9 (2.7%) |
| EAR AND LABYRINTH DISORDERS | 5 (3.0%) | 14 (4.3%) |
| HLGT: Inner ear and VIIIth cranial nerve disorders | 4 (2.4%) | 10 (3.0%) |
| HLT: Inner ear signs and symptoms | 4 (2.4%) | 10 (3.0%) |
| Tinnitus | 2 (1.2%) | 3 (0.9%) |
| Vertigo | 2 (1.2%) | 7 (2.1%) |
| CARDIAC DISORDERS | 14 (8.4%) | 21 (6.4%) |
| HLGT: Cardiac arrhythmias | 3 (1.8%) | 13 (4.0%) |
| HLT: Rate and rhythm disorders NEC | 1 (0.6%) | 6 (1.8%) |
| Tachycardia | 0 | 6 (1.8%) |
| HLGT: Coronary artery disorders | 11 (6.6%) | 6 (1.8%) |
| HLT: Coronary artery disorders NEC | 5 (3.0%) | 2 (0.6%) |
| Coronary artery disease | 5 (3.0%) | 2 (0.6%) |
| HLT: Ischaemic coronary artery disorders | 9 (5.4%) | 4 (1.2%) |
| Acute myocardial infarction | 2 (1.2%) | 1 (0.3%) |
| Angina pectoris | 5 (3.0%) | 2 (0.6%) |
| VASCULAR DISORDERS | 14 (8.4%) | 34 (10.4%) |
| HLGT: Vascular hypertensive disorders | 12 (7.2%) | 23 (7.0%) |
| HLT: Accelerated and malignant hypertension | 3 (1.8%) | 6 (1.8%) |
| Hypertensive crisis | 3 (1.8%) | 6 (1.8%) |
| HLT: Vascular hypertensive disorders NEC | 9 (5.4%) | 17 (5.2%) |
| Hypertension | 9 (5.4%) | 17 (5.2%) |
| RESPIRATORY, THORACIC AND | 18 (10.8%) | 39 (11.9%) |
| MEDIASTINAL DISORDERS | | |
| HLGT: Respiratory disorders NEC | 13 (7.8%) | 26 (7.9%) |
| HLT: Breathing abnormalities | 4 (2.4%) | 1 (0.3%) |
| Dyspnoea | 2 (1.2%) | 0 |
| Dyspnoea exertional | 2 (1.2%) | 1 (0.3%) |
| HLT: Coughing and associated symptoms | 3 (1.8%) | 14 (4.3%) |
| Cough | 3 (1.8%) | 14 (4.3%) |
| HLT: Upper respiratory tract signs and symptoms | 5 (3.0%) | 17 (5.2%) |
| Oropharyngeal pain | 2 (1.2%) | 13 (4.0%) |
| HLGT: Upper respiratory tract disorders (excl infections) | 2 (1.2%) | 12 (3.7%) |
| HLT: Paranasal sinus disorders (excl infections and neoplasms) | 0 | 4 (1.2%) |
| Sinus congestion | 0 | 4 (1.2%) |
| GASTROINTESTINAL DISORDERS | 43 (25.7%) | 153 (46.6%) |
| HLGT: Dental and gingival conditions | 7 (4.2%) | 12 (3.7%) |
| HLT: Dental pain and sensation disorders | 4 (2.4%) | 5 (1.5%) |
| Toothache | 4 (2.4%) | 5 (1.5%) |
| HLGT: Gastrointestinal inflammatory conditions | 4 (2.4%) | 4 (1.2%) |
| HLT: Gastritis (excl infective) | 2 (1.2%) | 4 (1.2%) |
| Gastritis | 2 (1.2%) | 3 (0.9%) |
| HLGT: Gastrointestinal motility and defaecation conditions | 14 (8.4%) | 52 (15.9%) |
| HLT: Diarrhoea (excl infective) | 10 (6.0%) | 38 (11.6%) |
| Diarrhoea | 10 (6.0%) | 37 (11.3%) |
| HLT: Gastrointestinal atonic and hypomotility disorders NEC | 4 (2.4%) | 19 (5.8%) |
| Constipation | 4 (2.4%) | 16 (4.9%) |
| Gastrooesophageal reflux disease | 0 | 4 (1.2%) |
| HLGT: Gastrointestinal signs and symptoms | 23 (13.8%) | 126 (38.4%) |
| HLT: Dyspeptic signs and symptoms | 1 (0.6%) | 17 (5.2%) |
| Dyspepsia | 1 (0.6%) | 17 (5.2%) |
| HLT: Flatulence, bloating and distension | 1 (0.6%) | 11 (3.4%) |
| Abdominal distension | 1 (0.6%) | 6 (1.8%) |
| Flatulence | 0 | 7 (2.1%) |
| HLT: Gastrointestinal and abdominal pains (excl oral and throat) | 5 (3.0%) | 21 (6.4%) |
| Abdominal pain | 2 (1.2%) | 9 (2.7%) |
| Abdominal pain upper | 3 (1.8%) | 13 (4.0%) |
| HLT: Gastrointestinal signs and symptoms NEC | 3 (1.8%) | 5 (1.5%) |
| Abdominal discomfort | 2 (1.2%) | 3 (0.9%) |
| HLT: Nausea and vomiting symptoms | 17 (10.2%) | 104 (31.7%) |
| Nausea | 16 (9.6%) | 96 (29.3%) |
| Vomiting | 2 (1.2%) | 32 (9.8%) |
| SKIN AND SUBCUTANEOUS TISSUE | 21 (12.6%) | 37 (11.3%) |
| DISORDERS | | |
| HLGT: Angioedema and urticaria | 0 | 6 (1.8%) |
| HLT: Urticarias | 0 | 6 (1.8%) |
| Urticaria | 0 | 6 (1.8%) |
| HLGT: Epidermal and dermal conditions | 11 (6.6%) | 17 (5.2%) |
| HLT: Dermatitis and eczema | 4 (2.4%) | 3 (0.9%) |
| Dermatitis | 2 (1.2%) | 0 |
| HLT: Pruritus NEC | 3 (1.8%) | 7 (2.1%) |
| Pruritus | 3 (1.8%) | 5 (1.5%) |
| HLT: Rashes, eruptions and exanthems NEC | 2 (1.2%) | 2 (0.6%) |
| Rash | 2 (1.2%) | 2 (0.6%) |
| HLGT: Skin appendage conditions | 8 (4.8%) | 13 (4.0%) |
| HLT: Apocrine and eccrine gland disorders | 5 (3.0%) | 11 (3.4%) |
| Cold sweat | 2 (1.2%) | 1 (0.3%) |
| Hyperhidrosis | 3 (1.8%) | 10 (3.0%) |
| HLT: Rosaceas | 2 (1.2%) | 0 |
| Rosacea | 2 (1.2%) | 0 |
| MUSCULOSKELETAL AND CONNECTIVE | 38 (22.8%) | 90 (27.4%) |
| TISSUE DISORDERS | | |
| HLGT: Joint disorders | 13 (7.8%) | 27 (8.2%) |
| HLT: Joint related signs and symptoms | 11 (6.6%) | 16 (4.9%) |
| Arthralgia | 11 (6.6%) | 16 (4.9%) |
| HLT: Osteoarthropathies | 2 (1.2%) | 9 (2.7%) |
| Osteoarthritis | 2 (1.2%) | 9 (2.7%) |
| HLGT: Muscle disorders | 7 (4.2%) | 16 (4.9%) |
| HLT: Muscle pains | 4 (2.4%) | 8 (2.4%) |
| Myalgia | 4 (2.4%) | 5 (1.5%) |
| HLT: Muscle related signs and symptoms NEC | 2 (1.2%) | 7 (2.1%) |
| Muscle spasms | 2 (1.2%) | 7 (2.1%) |
| HLGT: Musculoskeletal and connective tissue disorders NEC | 23 (13.8%) | 55 (16.8%) |
| HLT: Musculoskeletal and connective tissue pain and discomfort | 23 (13.8%) | 52 (15.9%) |
| Back pain | 11 (6.6%) | 24 (7.3%) |
| Musculoskeletal pain | 6 (3.6%) | 10 (3.0%) |
| Pain in extremity | 7 (4.2%) | 15 (4.6%) |
| HLGT: Tendon, ligament and cartilage disorders | 1 (0.6%) | 7 (2.1%) |
| HLT: Tendon disorders | 1 (0.6%) | 7 (2.1%) |
| Tendonitis | 1 (0.6%) | 6 (1.8%) |
| RENAL AND URINARY DISORDERS | 8 (4.8%) | 16 (4.9%) |
| HLGT: Nephropathies | 2 (1.2%) | 1 (0.3%) |
| HLT: Nephropathies and tubular disorders NEC | 2 (1.2%) | 1 (0.3%) |
| Diabetic nephropathy | 2 (1.2%) | 1 (0.3%) |
| HLGT: Renal disorders (excl nephropathies) | 3 (1.8%) | 3 (0.9%) |
| HLT: Renal failure and impairment | 3 (1.8%) | 3 (0.9%) |
| Renal failure | 2 (1.2%) | 0 |
| HLGT: Urinary tract signs and symptoms | 2 (1.2%) | 8 (2.4%) |
| HLT: Bladder and urethral symptoms | 1 (0.6%) | 4 (1.2%) |
| Dysuria | 1 (0.6%) | 4 (1.2%) |
| GENERAL DISORDERS AND ADMINISTRATION | 34 (20.4%) | 69 (21.0%) |
| SITE CONDITIONS | | |
| HLGT: Body temperature conditions | 3 (1.8%) | 5 (1.5%) |
| HLT: Febrile disorders | 3 (1.8%) | 5 (1.5%) |
| Pyrexia | 3 (1.8%) | 5 (1.5%) |
| HLGT: General system disorders NEC | 30 (18.0%) | 59 (18.0%) |
| HLT: Asthenic conditions | 17 (10.2%) | 34 (10.4%) |
| Asthenia | 10 (6.0%) | 18 (5.5%) |
| Fatigue | 6 (3.6%) | 15 (4.6%) |
| Malaise | 2 (1.2%) | 3 (0.9%) |
| HLT: Feelings and sensations NEC | 2 (1.2%) | 7 (2.1%) |
| Hunger | 2 (1.2%) | 2 (0.6%) |
| HLT: Oedema NEC | 7 (4.2%) | 12 (3.7%) |
| Oedema peripheral | 7 (4.2%) | 8 (2.4%) |
| HLT: Pain and discomfort NEC | 7 (4.2%) | 12 (3.7%) |
| Chest pain | 4 (2.4%) | 1 (0.3%) |
| Non-cardiac chest pain | 1 (0.6%) | 6 (1.8%) |
| Pain | 1 (0.6%) | 4 (1.2%) |
| INVESTIGATIONS | 20 (12.0%) | 38 (11.6%) |
| HLGT: Endocrine investigations (incl sex hormones) | 2 (1.2%) | 4 (1.2%) |
| HLT: Gastrointestinal, pancreatic and APUD hormone analyses | 2 (1.2%) | 4 (1.2%) |
| Blood calcitonin increased | 2 (1.2%) | 4 (1.2%) |
| HLGT: Gastrointestinal investigations | 2 (1.2%) | 6 (1.8%) |
| HLT: Digestive enzymes | 2 (1.2%) | 6 (1.8%) |
| Lipase increased | 2 (1.2%) | 5 (1.5%) |
| HLGT: Hepatobiliary investigations | 4 (2.4%) | 3 (0.9%) |
| HLT: Liver function analyses | 4 (2.4%) | 3 (0.9%) |
| Alanine aminotransferase increased | 2 (1.2%) | 2 (0.6%) |
| HLGT: Metabolic, nutritional and blood gas investigations | 8 (4.8%) | 20 (6.1%) |
| HLT: Carbohydrate tolerance analyses (incl diabetes) | 7 (4.2%) | 19 (5.8%) |
| Blood glucose decreased | 7 (4.2%) | 19 (5.8%) |
| INJURY, POISONING AND PROCEDURAL COMPLICATIONS | 16 (9.6%) | 43 (13.1%) |
| HLGT: Bone and joint injuries | 6 (3.6%) | 13 (4.0%) |
| HLT: Upper limb fractures and dislocations | 3 (1.8%) | 1 (0.3%) |
| Wrist fracture | 3 (1.8%) | 0 |
| HLGT: Injuries NEC | 11 (6.6%) | 29 (8.8%) |
| HLT: Muscle, tendon and ligament injuries | 4 (2.4%) | 3 (0.9%) |
| Muscle strain | 3 (1.8%) | 3 (0.9%) |
| HLT: Non-site specific injuries NEC | 3 (1.8%) | 16 (4.9%) |
| Fall | 2 (1.2%) | 12 (3.7%) |
| HLT: Site specific injuries NEC | 3 (1.8%) | 4 (1.2%) |
| Tooth fracture | 2 (1.2%) | 2 (0.6%) |
| HLT: Skin injuries NEC | 3 (1.8%) | 8 (2.4%) |
| Contusion | 2 (1.2%) | 6 (1.8%) |
| Skin laceration | 2 (1.2%) | 1 (0.3%) |
| HLGT: Procedural related injuries and complications NEC | 2 (1.2%) | 2 (0.6%) |
| HLT: Non-site specific procedural complications | 2 (1.2%) | 1 (0.3%) |
| Procedural pain | 2 (1.2%) | 1 (0.3%) |
| SURGICAL AND MEDICAL PROCEDURES | 8 (4.8%) | 9 (2.7%) |
| HLGT: Vascular therapeutic procedures | 5 (3.0%) | 1 (0.3%) |
| HLT: Arterial therapeutic procedures (excl aortic) | 5 (3.0%) | 1 (0.3%) |
| Coronary angioplasty | 2 (1.2%) | 0 |
| Coronary artery bypass | 2 (1.2%) | 1 (0.3%) |

TEAE: Treatment emergent adverse event,
SOC: System Organ Class,
HLGT: High Level Group Term,
HLT: High Level Therm,
PT: Preferred Term.
On-treatment period for the whole study = the time from the first dose of double-blind study medication up to 3 days after the last dose administration.
MedDRA version: 13.1.
n (%) = number and percentage of patients with at least one TEAE.
Note:
Table sorted by SOC internationally agreed order and HLGT, HLT, PT alphabetic order.
Only SOC with at least one PT ≥1% in at least one group are presented.

1. A method for improving glycemic control in an adult subject with type 2 diabetes mellitus, comprising administering to the subject a therapeutically effective amount of a pharmaceutical combination comprising:
(a) desPro36Exendin-4(1-39)-Lys6-NH2 (“lixisenatide”), and
(b) Gly(A21)-Arg(B31)-Arg(B32) human insulin (“insulin glargine”);
wherein the pharmaceutical combination is administered as an adjunct to diet and exercise.

2. The method of claim 1, wherein the pharmaceutical combination is administered at a dose of 15 units to 60 units insulin glargine and 10 μg to 20 μg lixisenatide subcutaneously once daily.

3. The method of claim 2, wherein the glycemic control in the subject is inadequately controlled on 30 to 60 units of basal insulin.

4. The method of claim 3, wherein the pharmaceutical combination is administered at a starting dose of 30 units insulin glargine and 10 μg lixisenatide subcutaneously once daily.

5. The method of claim 2, wherein the glycemic control in the subject is inadequately controlled on less than 30 units of basal insulin.

6. The method of claim 5, wherein the pharmaceutical combination is administered at a starting dose of 15 units insulin glargine and 5 μg lixisenatide subcutaneously once daily.

7. The method of claim 2, wherein the glycemic control in the subject is inadequately controlled on a GLP-1 receptor agonist.

8. The method of claim 7, wherein the pharmaceutical combination is administered at a starting dose of 15 units insulin glargine and 5 μg lixisenatide subcutaneously once daily.


SEQUENCE LISTING (2 sequences)

SEQ ID NO: 1 - protein, 44 aa, synthetic construct
  REGION 1..44: note = AVE0010, Synthetic or recombination peptide, derivative of Exendin-4
HGEGTFTSDLSKQMEEEAVRLFIEWLKNGGPSSGAPPSKKKKKK

SEQ ID NO: 2 - protein, 39 aa, synthetic construct
  REGION 1..39: note = Exendin-4
HGEGTFTSDLSKQMEEEAVRLFIEWLKNGGPSSGAPPPS